SEQUENCE LISTING (65 sequences)

SEQ ID NO: 1 - RNA, 22 nt, Artificial Sequence
  miR204-5p (RNA)
uucccuuugucauccuaugccu

SEQ ID NO: 2 - RNA, 22 nt, Artificial Sequence
  miR204-5p (RNA) complement
aggcauaggaugacaaagggaa

SEQ ID NO: 3 - DNA, 22 bp, Artificial Sequence
  miR204-5p (DNA)
ttccctttgtcatcctatgcct

SEQ ID NO: 4 - DNA, 22 bp, Artificial Sequence
  miR204-5p (DNA) complement
aggcataggatgacaaagggaa

SEQ ID NO: 5 - RNA, 21 nt, Artificial Sequence
  miR204-3p (RNA)
gcugggaaggcaaagggacgu

SEQ ID NO: 6 - RNA, 21 nt, Artificial Sequence
  miR204-3p (RNA) complement
acgucccuuugccuucccagc

SEQ ID NO: 7 - DNA, 21 bp, Artificial Sequence
  miR204-3p (DNA)
gctgggaaggcaaagggacgt

SEQ ID NO: 8 - DNA, 21 bp, Artificial Sequence
  miR204-3p (DNA) complement
acgtccctttgccttcccagc

SEQ ID NO: 9 - RNA, 8 nt, Artificial Sequence
  Stem 2 (RNA)
guauucug

SEQ ID NO: 10 - DNA, 8 bp, Artificial Sequence
  Stem 2 (DNA)
gtattctg

SEQ ID NO: 11 - RNA, 8 nt, Artificial Sequence
  Stem 2 complement (RNA)
cagaauac

SEQ ID NO: 12 - DNA, 8 bp, Artificial Sequence
  Stem 2 complement (DNA)
cagaatac

SEQ ID NO: 13 - RNA, 4 nt, Artificial Sequence
  Loop (RNA)
guca

SEQ ID NO: 14 - DNA, 4 bp, Artificial Sequence
  Loop (DNA)
gtca

SEQ ID NO: 15 - RNA, 18 nt, Artificial Sequence
  Stem 1 (RNA)
gacggcgcuaggaucauc

SEQ ID NO: 16 - DNA, 18 bp, Artificial Sequence
  Stem 1  (DNA)
gacggcgctaggatcatc

SEQ ID NO: 17 - DNA, 18 bp, Artificial Sequence
  Stem 1 complement (RNA)
gaugauccuagctccguc

SEQ ID NO: 18 - DNA, 18 bp, Artificial Sequence
  Stem 1 complement (DNA)
gatgatcctagctccgtc

SEQ ID NO: 19 - DNA, 3 bp, Artificial Sequence
  5′ Spacer (DNA/RNA)
aac

SEQ ID NO: 20 - DNA, 3 bp, Artificial Sequence
  3′ Spacer (DNA/RNA)
caa

SEQ ID NO: 21 - RNA, 8 nt, Artificial Sequence
  Linker (RNA)
aacaauac

SEQ ID NO: 22 - DNA, 8 bp, Artificial Sequence
  Linker (DNA)
aacaatac

SEQ ID NO: 23 - DNA, 138 bp, Artificial Sequence
  miR204-5p TD construct
aaggatccgacggcgctaggatcatcaacaggcataggatgacaaagggaacaagtattctggtcacagaatacaacaggcataggatgacaaagggaacaagatgatcctagcgccgtcttttttggaaaagcttaa

SEQ ID NO: 24 - DNA, 136 bp, Artificial Sequence
  miR416 Control construct
aaggatccgacggcgctaggatcatcaacggttcgtacgtacactgttcacaagtattctggtcacagaatacaacggttcgtacgtacactgttcacaagatgatcctagcgccgtcttttttggaaaagcttaa

SEQ ID NO: 25 - RNA, 7 nt, Artificial Sequence
  miR204-5p seed sequence
uucccuu

SEQ ID NO: 26 - RNA, 7 nt, Artificial Sequence
  miR204-3p seed sequence
gcuggga

SEQ ID NO: 27 - DNA, 147 bp, Artificial Sequence
  RNA complex_204-5p_subtype1
aactcgaggttcgatacaggggcatcaagaggcataggatgacaaagggaagaatttggaacgtcagttccaaaaagaagaatagaaggcataggatgacaaagggaagaagatgcccctgtatcgaacttttttggaactcgagaa

SEQ ID NO: 28 - DNA, 147 bp, Artificial Sequence
  RNA complex_204-5p_subtype2
aactcgagtgcgcgctttgtggatgtaagaggcataggatgacaaagggaagaaaccccagtgtcaactggggtaagaagaatagaaggcataggatgacaaagggaagaaacatccacaaagcgcgcattttttggaactcgagaa

SEQ ID NO: 29 - DNA, 147 bp, Artificial Sequence
  RNA complex_204-5p_subtype3
aactcgagaataactagacacaatcgaagaggcataggatgacaaagggaagaaaccttctggtcacagaaggtaagaagaatagaaggcataggatgacaaagggaagaacgattgtgtctagttattttttttggaactcgagaa

SEQ ID NO: 30 - DNA, 147 bp, Artificial Sequence
  RNA complex_204-5p_subtype4
aactcgaggtttttatatgccaacacaagaggcataggatgacaaagggaagaatacccgaagtcattcgggtaaagaagaatagaaggcataggatgacaaagggaagaagtgttggcatataaaaacttttttggaactcgagaa

SEQ ID NO: 31 - DNA, 147 bp, Artificial Sequence
  RNA complex_204-5p_subtype5
aaggatccccttgggcttgtagcctaaagaggcataggatgacaaagggaagaagttgagcggtcacgctcaacaagaagaatagaaggcataggatgacaaagggaagaataggctacaagcccaaggttttttggaactcgagaa

SEQ ID NO: 32 - DNA, 147 bp, Artificial Sequence
  RNA complex_204-5p_subtype6
aaggatcctgtggattttccaaaaggaagaggcataggatgacaaagggaagaaaagatcgtgtcaacgatcttaagaagaatagaaggcataggatgacaaagggaagaaccttttggaaaatccacattttttggaactcgagaa

SEQ ID NO: 33 - DNA, 147 bp, Artificial Sequence
  RNA complex_204-5p_subtype7
aaggatccaagtcagccttattggacaagaggcataggatgacaaagggaagaaagcaataggtcactattgctaagaagaatagaaggcataggatgacaaagggaagaagtccaataaggctgacttttttttggaactcgagaa

SEQ ID NO: 34 - DNA, 147 bp, Artificial Sequence
  RNA complex_204-5p_subtype8
aaggatcctacgaccgagttcgtagtaagaggcataggatgacaaagggaagaacagtgaaagtcatttcactgaagaagaatagaaggcataggatgacaaagggaagaaactacgaactcggtcgtattttttggaactcgagaa

SEQ ID NO: 35 - DNA, 147 bp, Artificial Sequence
  RNA complex_204-5p_subtype9
aaggatcccccggcgaaatactgcgaaagaggcataggatgacaaagggaagaatagcattcgtcagaatgctaaagaagaatagaaggcataggatgacaaagggaagaatcgcagtatttcgccgggttttttggaactcgagaa

SEQ ID NO: 36 - DNA, 147 bp, Artificial Sequence
  RNA complex_204-5p_subtype10
aaggatcctggtggggacactgagctaagaggcataggatgacaaagggaagaagtctcgcagtcatgcgagacaagaagaatagaaggcataggatgacaaagggaagaaagctcagtgtccccaccattttttggaactcgagaa

SEQ ID NO: 37 - DNA, 147 bp, Artificial Sequence
  RNA complex_204-5p_subtype11
aaggatcctaaacagatcaacacgctaagaggcataggatgacaaagggaagaaagttgggcgtcagcccaactaagaagaatagaaggcataggatgacaaagggaagaaagcgtgttgatctgtttattttttggaactcgagaa

SEQ ID NO: 38 - DNA, 147 bp, Artificial Sequence
  RNA complex_204-5p_subtype12
aaggatcccgtcctacttaagagagtaagaggcataggatgacaaagggaagaagtctatacgtcagtatagacaagaagaatagaaggcataggatgacaaagggaagaaactctcttaagtaggacgttttttggaactcgagaa

SEQ ID NO: 39 - DNA, 147 bp, Artificial Sequence
  RNA complex_204-5p_subtype13
aaggatcctgtgttccgcggagatcaaagaggcataggatgacaaagggaagaaacaaaaacgtcagtttttgtaagaagaatagaaggcataggatgacaaagggaagaatgatctccgcggaacacattttttggaactcgagaa

SEQ ID NO: 40 - DNA, 147 bp, Artificial Sequence
  RNA complex_204-5p_subtype14
aaggatcctcctgatgtaagcttacaaagaggcataggatgacaaagggaagaaaattattcgtcagaataattaagaagaatagaaggcataggatgacaaagggaagaatgtaagcttacatcaggattttttggaactcgagaa

SEQ ID NO: 41 - DNA, 147 bp, Artificial Sequence
  RNA complex_204-5p_subtype15
aaggatccctttaagtgggacgatggaagaggcataggatgacaaagggaagaagaacggctgtcaagccgttcaagaagaatagaaggcataggatgacaaagggaagaaccatcgtcccacttaaagttttttggaactcgagaa

SEQ ID NO: 42 - DNA, 147 bp, Artificial Sequence
  RNA complex_204-5p_subtype16
aaggatccactcttagtgtccttatgaagaggcataggatgacaaagggaagaaagatcgaagtcattcgatctaagaagaatagaaggcataggatgacaaagggaagaacataaggacactaagagtttttttggaactcgagaa

SEQ ID NO: 43 - DNA, 147 bp, Artificial Sequence
  RNA complex_204-5p_subtype17
aaggatccgcagcgatcgctcgtacaaagaggcataggatgacaaagggaagaacaatccaagtcattggattgaagaagaatagaaggcataggatgacaaagggaagaatgtacgagcgatcgctgcttttttggaactcgagaa

SEQ ID NO: 44 - DNA, 147 bp, Artificial Sequence
  RNA complex_204-5p_subtype18
aaggatcctactaagacagtatctccaagaggcataggatgacaaagggaagaagtctctaggtcactagagacaagaagaatagaaggcataggatgacaaagggaagaaggagatactgtcttagtattttttggaactcgagaa

SEQ ID NO: 45 - DNA, 147 bp, Artificial Sequence
  RNA complex_204-5p_subtype19
aaggatcctgcggcgcgactaaccgaaagaggcataggatgacaaagggaagaaggtcactcgtcagagtgaccaagaagaatagaaggcataggatgacaaagggaagaatcggttagtcgcgccgcattttttggaactcgagaa

SEQ ID NO: 46 - DNA, 147 bp, Artificial Sequence
  RNA complex_204-5p_subtype20
aaggatcctacctatatctccacgttaagaggcataggatgacaaagggaagaagacgaatcgtcagattcgtcaagaagaatagaaggcataggatgacaaagggaagaaaacgtggagatataggtattttttggaactcgagaa

SEQ ID NO: 47 - DNA, 147 bp, Artificial Sequence
  RNA complex_204-5p_subtype21
aaggatccgacgagtcgtagcgcagaaagaggcataggatgacaaagggaagaacagttgctgtcaagcaactgaagaagaatagaaggcataggatgacaaagggaagaatctgcgctacgactcgtcttttttggaactcgagaa

SEQ ID NO: 48 - DNA, 147 bp, Artificial Sequence
  RNA complex_204-5p_subtype22
aaggatccgtaaatcaccctactgccaagaggcataggatgacaaagggaagaaactcttatgtcaataagagtaagaagaatagaaggcataggatgacaaagggaagaaggcagtagggtgatttacttttttggaactcgagaa

SEQ ID NO: 49 - DNA, 147 bp, Artificial Sequence
  RNA complex_204-5p_subtype23
aaggatccctaagttgttaataattgaagaggcataggatgacaaagggaagaaatgtgccggtcacggcacataagaagaatagaaggcataggatgacaaagggaagaacaattattaacaacttagttttttggaactcgagaa

SEQ ID NO: 50 - DNA, 147 bp, Artificial Sequence
  RNA complex_204-5p_subtype24
aaggatcccagaccagccagatcggcaagaggcataggatgacaaagggaagaacgcgaacggtcacgttcgcgaagaagaatagaaggcataggatgacaaagggaagaagccgatctggctggtctgttttttggaactcgagaa

SEQ ID NO: 51 - DNA, 147 bp, Artificial Sequence
  RNA complex_204-5p_subtype25
aaggatcctgcgtaacgaagtgggggaagaggcataggatgacaaagggaagaatagaggaggtcactcctctaaagaagaatagaaggcataggatgacaaagggaagaacccccacttcgttacgcattttttggaactcgagaa

SEQ ID NO: 52 - DNA, 147 bp, Artificial Sequence
  RNA complex_204-5p_subtype26
aaggatcccacggtcctagtcttttgaagaggcataggatgacaaagggaagaatcgtctaagtcattagacgaaagaagaatagaaggcataggatgacaaagggaagaacaaaagactaggaccgtgttttttggaactcgagaa

SEQ ID NO: 53 - DNA, 147 bp, Artificial Sequence
  RNA complex_204-5p_subtype27
aaggatccatagactgcggaggcggtaagaggcataggatgacaaagggaagaatacaaactgtcaagtttgtaaagaagaatagaaggcataggatgacaaagggaagaaaccgcctccgcagtctatttttttggaactcgagaa

SEQ ID NO: 54 - DNA, 147 bp, Artificial Sequence
  RNA complex_204-5p_subtype28
aaggatcctcaggtttaaaccaacctaagaggcataggatgacaaagggaagaaactgacctgtcaaggtcagtaagaagaatagaaggcataggatgacaaagggaagaaaggttggtttaaacctgattttttggaactcgagaa

SEQ ID NO: 55 - DNA, 147 bp, Artificial Sequence
  RNA complex_204-5p_subtype29
aaggatcctctgacatacttggggaaaagaggcataggatgacaaagggaagaaacttacgagtcatcgtaagtaagaagaatagaaggcataggatgacaaagggaagaattccccaagtatgtcagattttttggaactcgagaa

SEQ ID NO: 56 - DNA, 147 bp, Artificial Sequence
  RNA complex_204-5p_subtype30
aaggatccaagagttaagtagcctgaaagaggcataggatgacaaagggaagaaatcaataggtcactattgataagaagaatagaaggcataggatgacaaagggaagaatcaggctacttaactcttttttttggaactcgagaa

SEQ ID NO: 57 - DNA, 138 bp, Artificial Sequence
  RNA complex_204-5p_subtype31
aaggatccgacggcgctaggatcatcaacaggcataggatgacaaagggaacaagtattctggtcacagaatacaacaggcataggatgacaaagggaacaagatgatcctagcgccgtcttttttggaactcgagaa

SEQ ID NO: 58 - DNA, 1307 bp, Artificial Sequence
  nuclear receptor subfamily 4 group A member 2 (NR4A2)
gacctcctcccaagcacttcaaaggaactggaatgataatggaaactgtcaagagggggcaagtcacatgggcagagatagccgtgtgagcagtctcagctcaagctgccccccatttctgtaaccctcctagcccccttgatccctaaagaaaacaaacaaacaaacaaaaactgttgctatttcctaacctgcaggcagaacctgaaagggcattttggctccggggcatcctggatttagaacatggactacacacaatacagtggtataaactttttattctcagtttaaaaatcagtttgttgttcagaagaaagattgctataatgtataatgggaaatgtttggccatgcttggttgttgcagttcagacaaatgtaacacacacacacatacacacacacacacacacacagagacacatcttaaggggacccacaagtattgccctttaacaagacttcaaagttttctgctgtaaagaaagctgtaatatatagtaaaactaaatgttgcgtgggtggcatgagttgaagaaggcaaaggcttgtaaatttacccaatgcagtttggctttttaaattattttgtgcctatttatgaataaatattacaaattctaaaagataagtgtgtttgcaaaaaaaaagaaaataaatacataaaaaagggacaagcatgttgattctaggttgaaaatgttataggcacttgctacttcagtaatgtctatattatataaatagtatttcagacactatgtagtctgttagattttataaagattggtagttatctgagcttaaacattttctcaattgtaaaataggtgggcacaagtattacacatcagaaaatcctgacaaaagggacacatagtgtttgtaacaccgtccaacattccttgtttgtaagtgttgtatgtaccgttgatgttgataaaaagaaagtttatatcttgattattttgttgtctaaagctaaacaaaacttgcatgcagcagcttttgactgtttccagagtgcttataatatacataactccctggaaataactgagcactttgaattttttttatgtctaaaattgtcagttaatttattattttgtttgagtaagaattttaatattgccatattctgtagtatttttctttgtatatttctagtatggcacatgatatgagtcactgcctttttttctatggtgtatgacagttagagatgctgattttttttctgataaattctttctttgagaaagacaattttaatgtttacaacaataaaccatgtaaatgaa

SEQ ID NO: 59 - DNA, 25 bp, Artificial Sequence
  Nurr1
atcctgacaaaagggacacatagtg

SEQ ID NO: 60 - DNA, 26 bp, Artificial Sequence
  Nurr1
aatacataaaaaagggacaagcatgt

SEQ ID NO: 61 - DNA, 26 bp, Artificial Sequence
  Nurr1
aagcacttcaaaggaactggaatgat

SEQ ID NO: 62 - RNA, 22 nt, Artificial Sequence
  synthetic
uccguauccuacuguuucccuu

SEQ ID NO: 63 - protein, 598 aa, Artificial Sequence
  Isoform
MPCVQAQYGSSPQGASPASQSYSYHSSGEYSSDFLTPEFVKFSMDLTNTEITATTSLPSFSTFMDNYSTGYDVKPPCLYQMPLSGQQSSIKVEDIQMHNYQQHSHLPPQSEEMMPHSGSVYYKPSSPPTPTTPGFQVQHSPMWDDPGSLHNFHQNYVATTHMIEQRKTPVSRLSLFSFKQSPPGTPVSSCQMRFDGPLHVPMNPEPAGSHHVVDGQTFAVPNPIRKPASMGFPGLQIGHASQLLDTQVPSPPSRGSPSNEGLCAVCGDNAACQHYGVRTCEGCKGFFKRTVQKNAKYVCLANKNCPVDKRRRNRCQYCRFQKCLAVGMVKEVVRTDSLKGRRGRLPSKPKSPQEPSPPSPPVSLISALVRAHVDSNPAMTSLDYSRFQANPDYQMSGDDTQHIQQFYDLLTGSMEIIRGWAEKIPGFADLPKADQDLLFESAFLELFVLRLAYRSNPVEGKLIFCNGVVLHRLQCVRGFGEWIDSIVEFSSNLQNMNIDISAFSCIAALAMVTERHGLKEPKRVEELQNKIVNCLKDHVTFNNGGLNRPNYLSKLLGKLPELRTLCTQGLQRIFYLKLEDLVPPPAIIDKLFLDTLPF

SEQ ID NO: 64 - DNA, 147 bp, Artificial Sequence; identical to SEQ ID NO: 27
  synthetic

SEQ ID NO: 65 - DNA, 145 bp, Artificial Sequence
  synthetic
aactcgaggttcgatacaggggcatcaagaagaggcttgcacagtgcattgaatttggaacgtcagttccaaaaagaagaatagaaagaggcttgcacagtgcattgaagatgcccctgtatcgaacttttttggaactcgagaa

(19) United States
(12) Patent Application Publication
(10) Pub. No.: US 2022/0081690 A1
(43) Pub. Date: Mar. 17, 2022

(54) USE OF MIR-204 INHIBITOR TO INCREASE NURR1 PROTEIN EXPRESSION

(71) Applicant: BIORCHESTRA CO., LTD., Daejeon (KR)

(72) Inventors: Jin-Hyeob RYU, Daejeon (KR); Sang Moo LEE, Daejeon (KR)

(73) Assignee: BIORCHESTRA CO., LTD., Daejon (KR)

(21) Appl. No.: 17/259,291

(22) PCT Filed: Jun. 3, 2020
(86) PCT No.: PCT/IB2020/055253

§ 371 Date: Jan. 11, 2021

Related U.S. Application Data
(60) Provisional application No. 62/857,202, filed on Jun. 4, 2019.

(51) Int. Cl. C12N 15/113; C12N 15/86; A61P 25/28

(52) U.S. Cl. CPC C12N 15/113; C12N 15/86; A61P 25/28; C12N 2320/32; C12N 2750/14143; C12N 2310/13; C12N 2310/113

(57) ABSTRACT

The present disclosure includes use of a vector for treating a disease or condition associated with a decreased level of a Nurr1 protein. The vector useful for the present disclosure comprises a promoter and an RNA expression region, wherein the RNA expression region is located downstream of the promoter, wherein the RNA expression region comprises a nucleotide sequence expressing an RNA comprising at least one miR-204 binding site, and wherein the RNA expression region does not encode a protein.

### CROSS-REFERENCE TO RELATED APPLICATIONS

This PCT application claims the priority benefit of U.S. Provisional Application No. 62/857,202, filed Jun. 4, 2019, which is incorporated herein by reference in its entirety.

### REFERENCE TO SEQUENCE LISTING SUBMITTED ELECTRONICALLY

The content of the electronically submitted sequence listing in ASCII text file (Name 4366_001PC01_SequenceListing_ST25.txt; Size: 25,061 bytes; and Date of Creation: Jun. 3, 2020) filed with the application is incorporated herein by reference in its entirety.

### FIELD

The present disclosure provides use of a miR-204 inhibitor, e.g., viral vector capable of producing an RNA comprising at least one microRNA (miR) 204 binding site, for the treatment of neurodegenerative disorders associated with decreased expression of a Nurr1 protein.

### BACKGROUND ART

MicroRNAs (miRNAs or miRs) are an abundant class of short endogenous RNAs that act as post-transcriptional regulators of gene expression by base-pairing with their target mRNAs. The mature miRNAs are processed sequentially from longer hairpin transcripts by the RNAse III ribonucleases Drosha and Dicer. Most animal miRNAs recognize their target sites located in 3′-UTRs by incomplete base-pairing, resulting in translational repression of the target genes. An increasing body of research shows that animal miRNAs play fundamental biological roles in cell growth and apoptosis, hematopoietic lineage differentiation, life-span regulation, photoreceptor differentiation, homeobox gene regulation, neuronal asymmetry, insulin secretion, brain morphogenesis, muscle proliferation and differentiation, cardiogenesis, and late embryonic development.

miRNAs are involved in a wide variety of human diseases. For example, miRNAs are involved in spinal muscular atrophy (SMA), Tourette's syndrome, fragile X mental retraction, DiGeorge syndrome

Despite advances in diagnosis and treatment of the symptoms of neurodegenerative diseases such as Alzheimer's disease and Parkinson's disease, their prognosis is still poor. MicroRNAs can regulate gene expression in cells and be used therapeutically. A difficulty to be overcome for effective therapy using miRNA is the efficient administration of therapeutic miRNA to cells, tissues, or organs.

### BRIEF SUMMARY

In some aspects, the present disclosure is directed to a method of treating a disease or condition associated with a decreased level of a Nurr1 protein in a subject in need thereof comprising administering a miR-204 inhibitor. In other aspects, the present disclosure provides a method of increasing a Nurr1 protein expression in a cell comprising contacting the cell with a miR-204 inhibitor. In some aspects, the cell is present in a subject.

In certain aspects, the miR-204 inhibitor comprises a nucleotide sequence comprising at least one miR-204 binding site. In other aspects, the nucleotide sequence is a vector comprising a promoter and an RNA expression region. For example, the RNA expression region can be located downstream of the promoter, wherein the RNA expression region comprises a nucleotide sequence expressing an RNA comprising at least one miR-204 binding site, and wherein the RNA expression region does not encode a protein. In other aspects, the vector does not encode a protein that is heterologous to the vector.

In some aspects, the at least one miR-204 binding site binds to endogenous miR-204 and regulates expression of one or more endogenous polypeptides. In other aspects, the at least one miR204 binding site increases expression of the Nurr1 protein.

In other aspects, the miR204 inhibitor useful for the disclosure does not increase expression of an NMDA receptor. In other aspects, the miR204 inhibitor does not increase expression of a EphB2 protein. In yet other aspects, the miR204 inhibitor increases the expression of the Nurr1 protein after the administration or contact by at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, at least about 6 fold, at least about 6.5 fold, at least about 7 fold, at least about 7.5 fold, or at least about 8 fold compared to the expression prior to the administration or contact.

In some aspects, the miR204 inhibitor treats a disease or condition associated with a decreased expression of the Nurr1 protein, but not with a decreased expression of an NMDA receptor and/or an EphB2 protein. In other aspects, the disease or condition is not associated with a decreased hippocampus function. In other aspects, the disease or condition is Parkinson's disease, prion disease, motor neuron disease, Huntington's disease, spinocerebellar ataxia, spinal muscular atrophy, amyotrophic lateral sclerosis, or any combination thereof.

In some aspects, the at least one miR-204 binding site hybridizes to miR-204-5p. In some aspects, the at least one miR-204 binding site is fully complementary to miR-204-5p. In some aspects, the miR-204-5p comprises the nucleotide sequence as set forth in SEQ ID NO: 1. In some aspects, the at least one miR-204 binding site comprises the nucleic acid sequence set forth in SEQ ID NO: 2. In some aspects, the nucleotide sequence expressing the RNA comprises the nucleic acid sequence set forth in SEQ ID NO: 3.

In some aspects, the at least one miR-204 binding site hybridizes to miR-204-3p. In some aspects, the at least one miR-204 binding site is fully complementary to miR-204-3p. In some aspects, the miR-204-3p comprises the nucleotide sequence as set forth in SEQ ID NO: 5. In some aspects, the at least one miR-204 binding site comprises the nucleic acid sequence set forth in SEQ ID NO: 6. In some aspects, the nucleotide sequence expressing the RNA comprises the nucleic acid sequence set forth in SEQ ID NO: 7.

In some aspects, the RNA comprises at least two miR-204 binding sites. In some aspects, the RNA comprises two miR-204 binding sites, three miR-204 binding sites, four miR-204 binding sites, five miR-204 binding sites, or six miR-204 binding sites. In some aspects, the RNA comprises two miR-204 binding sites. In some aspects, each of the at least one miR-204 binding site comprises the nucleic acid sequence set forth in SEQ ID NO:19 at the 5′ end. In some aspects, each of the at least one miR-204 binding site comprises the nucleic acid sequence set forth in SEQ ID NO:20 at the 3′ end. In some aspects, the two miR-204 binding sites comprise a nucleotide sequence forming a loop in between the miR-204 binding sites. In some aspects, the loop comprises a nucleotide sequence comprising the nucleic acid sequence set forth in SEQ ID NO:13. In some aspects, the RNA comprising the two miR-204 binding sites comprises a first stem region and a second stem region. In some aspects, the first stem region comprises a nucleotide sequence set forth in SEQ ID NO:9 or its complementary nucleotide sequence set forth in SEQ ID NO:11, which is linked to at least one of the two miR-204 binding sites. In some aspects, the second stem region comprises a nucleotide sequence of set forth in SEQ ID NO:15 or its complementary nucleotide sequence set forth in SEQ ID NO:17, which is linked to at least one of the two miR-204 binding sites. In some aspects, the RNA comprises the nucleic acid sequence set forth in SEQ ID NO: 23, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57.

In some aspects, the vector of the disclosure is a virus, a plasmid, or a phagemid. In some aspects, the vector of the disclosure is a virus. In some aspects, the virus is selected from the group consisting of a retrovirus, a lentivirus, an adenovirus, an adeno-associated virus (AAV), an SV40-type virus, a polyomavirus, an Epstein-Barr virus, a papilloma viruses, a herpes virus, a vaccinia virus, a polio virus, and an RNA virus. In some aspects, the vector is an AAV. In some aspects, the AAV is selected from the group consisting of AAV type 1, AAV type 2, AAV type 3A, AAV type 3B, AAV type 4, AAV type 5, AAV type 6, AAV type 7, AAV type 8, AAV type 9, AAV type 10, AAV type 11, AAV type 12, AAV type 13, snake AAV, avian AAV, bovine AAV, canine AAV, equine AAV, ovine AAV, goat AAV, shrimp AAV, and a derivative thereof.

In some aspects, the promoter is an RNA Pol III promoter. In some aspects, the RNA Pol III promoter is selected from the group consisting of the U6 promoter, the H1 promoter, the 7SK promoter, the 5S promoter, the adenovirus 2 (Ad2) VAI promoter, and any combination thereof. In some aspects, the promoter comprises the U6 promoter. In some aspects, the promoter is a constitutive promoter. In some aspects, the constitutive promoter is selected from the group consisting of hypoxanthine phosphoribosyl transferase (HPRT), adenosine deaminase, pyruvate kinase, beta-actin promoter, cytomegalovirus (CMV), simian virus (e.g., SV40), papilloma virus, adenovirus, human immunodeficiency virus (HIV), Rous sarcoma virus, a retrovirus long terminal repeat (LTR), and the thymidine kinase promoter of herpes simplex virus. In some aspects, the promoter is an inducible promoter. In some aspects, the inducible promoter is a tissue specific promoter. In some aspects, the tissue specific promoter drives transcription of the coding region in a neuron, a glial cell, or in both a neuron and a glial cell.

In some aspects, the miR204 inhibitor is formulated with a pharmaceutically acceptable carrier in a pharmaceutical composition. In some aspects, the administering improves one or more cognitive symptom in the subject, relative to the cognitive symptom in the subject prior to the administering. In some aspects, the administering reduces memory loss in the subject, relative to the memory loss in the subject prior to the administering. In some aspects, the administering improves memory retention in the subject, relative to the memory retention in the subject prior to the administering. In some aspects, the administering reduces an amyloid beta (Aβ) plaque load in the subject, relative to the amyloid beta (Aβ) plaque load in the subject prior to the administering. In some aspects, the administering increases dendritic spine density of a neuron in the subject, relative to the dendritic spine density of a neuron in the subject prior to the administering.

In some aspects, the administering is via intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

### BRIEF DESCRIPTION OF THE DRAWINGS

FIG. 1A shows an analysis of mRNA microarray data from 4 age-matched controls and 4 AD patients. The expression data are available in the NCBI gene expression omnibus (GEO) as accession number GES16759.

FIG. 1B shows comparison of the Nurr1 mRNA between AD patients' tissues and the tissues of non-AD patients.

FIG. 2A shows an analysis of mRNA microarray data from 60 AD patients' temporal cortical brain. The expression data are available NCBI's Gene Expression Omnibus (Edgar, 2002) and are accessible through GEO Series accession number GSE106241. FIG. 2A shows the Braak stages of samples.

FIG. 2B shows the comparison data of the Nurr1 mRNA expression between the AD patients: one group with Braak 0-3 and another group with Braak 4-6.

FIG. 3 shows the clinical data of mass spectrometry proteomics from ProteomeXchange Consortium via the PRIDE partner repository with the dataset identifier PXD008016B.

FIG. 4 shows an analysis of mRNA microarray data from 60 AD patients' temporal cortical brain. The microarray data have been deposited in NCBI's Gene Expression Omnibus (Edgar, 2002) and are accessible through GEO Series accession number GSE106241. It shows that Nurr1 expression pattern is dependent on APOE4 genotype.

FIG. 5 shows an analysis of mass spectrometry proteomics data from 60 AD patients' temporal cortical brain dataset. The dataset is identified with identifier PXD008016. The Nurr1 mRNA expression pattern (Y axis) is dependent on the level of Amyloid β protein. The microarray data discussed in this publication have been deposited in NCBI's Gene Expression Omnibus (Edgar, 2002) and are accessible through GEO Series accession number GSE106241. The mass spectrometry proteomics data have been deposited to the ProteomeXchange Consortium via the PRIDE partner repository with the dataset identifier PXD008016 (Vizcaino et al., 2016).

FIG. 6 shows an analysis of mass spectrometry proteomics data from 60 AD patients' temporal cortical brain dataset. The dataset is identified with identifier PXD008016. The Nurr1 mRNA expression pattern (Y axis) is dependent on the β secretase level (X axis).

FIG. 7 shows the ′UTR of Homo sapiens nuclear receptor subfamily 4 group A member 2 (NR4A2), transcript variant 1, mRNA. The underlined sequences is the sequence that binds to the sequence of miR-204-5p. See FIG. 8.

FIG. 8 shows the miR-204-5p seed sequence aligned with a portion of the Nurr1 3′ UTR region.

FIG. 9 shows the comparison of the luciferase activity expressed from each vector containing the wild type 3′ UTR of human Nurr1 (left two bars) or the mutant 3′ UTR of human Nurr1 (right two bars) when pCMV-miR-204-5p expressing a miR-204-5p binding site was added to each vector.

FIG. 10 shows a mechanism of an anti-miR204-5p inhibitor. An anti-miR-204-5p can prevent the interaction between miR-204-5p with the 3′ UTR of Nurr1, thereby increasing the expression of the Nurr1 protein.

FIG. 11 shows an immunoblot of Nurr1 proteins in cell lysates of control Mock- or Anti-miR-204-5p-treated primary neuron culture cell lines. Anti-miR 204-5p increases Nurr1 expression in primary neuron culture cell lines.

FIG. 12 shows a schematic of AAV viral therapeutic system.

FIG. 13 shows the representative cortical images from confocal imaging of Nurr1. The left panels show the 5×FAD mice cortex after administered with a negative control; The right panels show the 5×FAD mice cortex after administered with a viral system expressing anti-miR-204.

FIGS. 14A and 14B show immunoblot detection of Nurr1 proteins in brain lysates of control Mock- or Viral system anti-miR-204-5p-treated 5×FAD. Viral system anti-miR 204-5p promotes Nurr1 expression in 5×FAD brain.

FIG. 15 shows an immunohistochemical analysis of dentate gyrus of 5×FAD. Viral system anti-miR-204 decreases amyloid plaque burden in 5×FAD. Immunohistochemical analysis of dentate gyrus after administration of mock or Viral system anti-miR-204. Diffuse plaques in the brain sections were stained by anti-amyloid beta (clone 6E10, red color) and nucleus (blue).

FIG. 16 shows the results of the novel objective recognition test after administration of a viral system expressing an anti-miR-204 inhibitor and a negative control.

FIG. 17 shows exemplary Tough Decoys architectures comprising 1, 2, 3, 4 or 5 microRNA binding sites. The top left diagram show the modular structure of Tough Decoys comprising two or more mRNA binding sites (MBS).

### DETAILED DESCRIPTION

The present disclosure is directed to use of a miR-204 inhibitor, e.g., vectors, e.g., AAV vectors, comprising a promoter and an RNA expression region located, e.g., downstream from the promoter, wherein the RNA expression region comprises a nucleotide sequence encoding an RNA comprising at least one miRNA-204 binding site, wherein the RNA expression region does not encode a protein. The miR-204 binding site or sites can bind to endogenous miR-204, regulating expression of one or more endogenous polypeptides, which in turns treats or ameliorate the symptoms of a neurodegenerative disease, e.g., Alzheimer's disease or Parkinson's disease. Non-limiting examples of various aspects are shown in the present disclosure.

Before the present disclosure is described in greater detail, it is to be understood that this disclosure is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present disclosure. Any recited method can be carried out in the order of events recited or in any other order which is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

Accordingly, the terms defined immediately below are more fully defined by reference to the specification in its entirety.

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, ‘a’ represents adenine, ‘c’ represents cytosine, ‘g’ represents guanine, ‘t’ represents thymine, and ‘u’ represents uracil.

Amino acid sequences are written left to right in amino to carboxy orientation. Amino acids are referred to herein by either their commonly known three letter symbols or by the one-letter symbols recommended by the IUPAC-IUB Biochemical Nomenclature Commission.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAVrh.74, snake AAV, avian AAV, bovine AAV, canine AAV, equine AAV, ovine AAV, goat AAV, shrimp AAV, those AAV serotypes and clades disclosed by Gao et al. (J. Virol. 78:6381 (2004)) and Moris et al. (Virol. 33:375 (2004)), and any other AAV now known or later discovered. See, e.g., FIELDS et al. VIROLOGY, volume 2, chapter 69 (4th ed., Lippincott-Raven Publishers). In some embodiments, an “AAV” includes a derivative of a known AAV. In some embodiments, an “AAV” includes a modified or an artificial AAV.

The terms “administration,” “administering,” and grammatical variants thereof refer to introducing a composition, such as a vector of the present disclosure, into a subject via a pharmaceutically acceptable route. The introduction of a composition, such as a vector of the present disclosure, into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. Administration includes self-administration and the administration by another. A suitable route of administration allows the composition or the agent to perform its intended function. For example, if a suitable route is intravenous, the composition is administered by introducing the composition or agent into a vein of the subject.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are at least 70% identical, at least 80% identical, at least 90% identical, or at least 95% identical to one another. In some aspects, two or more sequences are said to be “highly conserved” if they are about 70% identical, about 80% identical, about 90% identical, about 95%, about 98%, or about 99% identical to one another. In some aspects, two or more sequences are said to be “conserved” if they are at least 30% identical, at least 40% identical, at least 50% identical, at least 60% identical, at least 70% identical, at least 80% identical, at least 90% identical, or at least 95% identical to one another. In some aspects, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an polynucleotide or polypeptide or may apply to a portion, region or feature thereof.

The term “derived from,” as used herein, refers to a component that is isolated from or made using a specified molecule or organism, or information (e.g., amino acid or nucleic acid sequence) from the specified molecule or organism. For example, a nucleic acid sequence that is derived from a second nucleic acid sequence can include a nucleotide sequence that is identical or substantially similar to the nucleotide sequence of the second nucleic acid sequence. In the case of nucleotides or polypeptides, the derived species can be obtained by, for example, naturally occurring mutagenesis, artificial directed mutagenesis or artificial random mutagenesis. The mutagenesis used to derive nucleotides or polypeptides can be intentionally directed or intentionally random, or a mixture of each. The mutagenesis of a nucleotide or polypeptide to create a different nucleotide or polypeptide derived from the first can be a random event (e.g., caused by polymerase infidelity) and the identification of the derived nucleotide or polypeptide can be made by appropriate screening methods, e.g., as discussed herein. Mutagenesis of a polypeptide typically entails manipulation of the polynucleotide that encodes the polypeptide. In some embodiments, a nucleotide or amino acid sequence that is derived from a second nucleotide or amino acid sequence has a sequence identity of at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% to the second nucleotide or amino acid sequence, respectively, wherein the first nucleotide or amino acid sequence retains the biological activity of the second nucleotide or amino acid sequence.

As used herein, an “RNA expression region” or “RNA expression sequence” refers to a polynucleotide sequence that can transcribe into an RNA sequence. Unless otherwise indicated, the RNA expression region as used in the presents disclosure does not translate into amino acids, but remains as RNAs only. The RNA expression region can be operably linked to a promoter and a termination sequence.

As used herein, a “coding region” or “coding sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide.

The terms “complementary” and “complementarity” refer to two or more oligomers (i.e., each comprising a nucleobase sequence), or between an oligomer and a target gene, that are related with one another by Watson-Crick base-pairing rules. For example, the nucleobase sequence “T-G-A (5′→3′),” is complementary to the nucleobase sequence “A-C-T (3′→5′).” Complementarity may be “partial,” in which less than all of the nucleobases of a given nucleobase sequence are matched to the other nucleobase sequence according to base pairing rules. For example, in some embodiments, complementarity between a given nucleobase sequence and the other nucleobase sequence may be about 70%, about 75%, about 80%, about 85%, about 90% or about 95%. Or, there may be “complete” or “perfect” (100%) complementarity between a given nucleobase sequence and the other nucleobase sequence to continue the example. The degree of complementarity between nucleobase sequences has significant effects on the efficiency and strength of hybridization between the sequences.

The term “downstream” refers to a nucleotide sequence that is located 3′ to a reference nucleotide sequence. In certain embodiments, downstream nucleotide sequences relate to sequences that follow the starting point of transcription. For example, the translation initiation codon of a gene is located downstream of the start site of transcription.

The terms “excipient” and “carrier” are used interchangeably and refer to an inert substance added to a pharmaceutical composition to further facilitate administration of a compound.

The term “expression” as used herein refers to a process by which a polynucleotide produces a gene product, for example, an RNA. It includes without limitation transcription of the polynucleotide into micro RNA binding site, small hairpin RNA (shRNA), small interfering RNA (siRNA) or any other RNA product. Expression produces a “gene product.” As used herein, a gene product can be, e.g., a nucleic acid, such as an RNA produced by transcription of a gene. Gene products described herein further include nucleic acids with post transcriptional modifications, e.g., polyadenylation. The term “yield,” as used herein, refers to the amount of a gene product produced by the expression of a gene.

As used herein, the term “homology” refers to the overall relatedness between polymeric molecules, e.g. between nucleic acid molecules (e.g. DNA molecules and/or RNA molecules) and/or between polypeptide molecules. Generally, the term “homology” implies an evolutionary relationship between two molecules. Thus, two molecules that are homologous will have a common evolutionary ancestor. In the context of the present disclosure, the term homology encompasses both to identity and similarity.

In some aspects, polymeric molecules are considered to be “homologous” to one another if at least 25%, 30%, 35%, 40%, 45%, 50%, 55%, 60%, 65%, 70%, 75%, 80%, 85%, 90%, 95%, or 99% of the monomers in the molecule are identical (exactly the same monomer) or are similar (conservative substitutions). The term “homologous” necessarily refers to a comparison between at least two sequences (polynucleotide or polypeptide sequences).

In the context of the present disclosure, substitutions (even when they are referred to as amino acid substitution) are conducted at the nucleic acid level, i.e., substituting an amino acid residue with an alternative amino acid residue is conducted by substituting the codon encoding the first amino acid with a codon encoding the second amino acid.

As used herein, the term “identity” refers to the overall monomer conservation between polymeric molecules, e.g., between polypeptide molecules or polynucleotide molecules (e.g. DNA molecules and/or RNA molecules). The term “identical” without any additional qualifiers, e.g., protein A is identical to protein B, implies the sequences are 100% identical (100% sequence identity). Describing two sequences as, e.g., “70% identical,” is equivalent to describing them as having, e.g., “70% sequence identity.”

Calculation of the percent identity of two polypeptide sequences, for example, can be performed by aligning the two sequences for optimal comparison purposes (e.g., gaps can be introduced in one or both of a first and a second polypeptide sequences for optimal alignment and non-identical sequences can be disregarded for comparison purposes). In certain aspects, the length of a sequence aligned for comparison purposes is at least 30%, at least 40%, at least 50%, at least 60%, at least 70%, at least 80%, at least 90%, at least 95%, or 100% of the length of the reference sequence. The amino acids at corresponding amino acid positions are then compared.

When a position in the first sequence is occupied by the same amino acid as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

As used herein, the terms “isolated,” “purified,” “extracted,” and grammatical variants thereof are used interchangeably and refer to the state of a preparation of desired vector of the present disclosure, that has undergone one or more processes of purification. In some aspects, isolating or purifying as used herein is the process of removing, partially removing (e.g., a fraction) of a composition comprising a vector of the present disclosure from a sample containing cells. In some aspects, an isolated composition has no detectable undesired activity or, alternatively, the level or amount of the undesired activity is at or below an acceptable level or amount. In other aspects, an isolated composition has an amount and/or concentration of desired vector of the present disclosure, at or above an acceptable amount and/or concentration and/or activity. In other aspects, the isolated composition is enriched as compared to the starting material (e.g., cell preparation) from which the composition is obtained. This enrichment can be by at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.9%, at least about 99.99%, at least about 99.999%, at least about 99.9999%, or greater than 99.9999% as compared to the starting material. In some aspects, isolated preparations are substantially free of residual biological products. In some aspects, the isolated preparations are 100% free, at least about 99% free, at least about 98% free, at least about 97% free, at least about 96% free, at least about 95% free, at least about 94% free, at least about 93% free, at least about 92% free, at least about 91% free, or at least about 90% free of any contaminating biological matter. Residual biological products can include abiotic materials (including chemicals) or unwanted nucleic acids, proteins, lipids, or metabolites.

The term “linked” as used herein refers to a first amino acid sequence or polynucleotide sequence covalently or non-covalently joined to a second amino acid sequence or polynucleotide sequence, respectively. The first amino acid or polynucleotide sequence can be directly joined or juxtaposed to the second amino acid or polynucleotide sequence or alternatively an intervening sequence can covalently join the first sequence to the second sequence. The term “linked” means not only a fusion of a first polynucleotide sequence to a second polynucleotide sequence at the 5′-end or the 3′-end, but also includes insertion of the whole first polynucleotide sequence (or the second polynucleotide sequence) into any two nucleotides in the second polynucleotide sequence (or the first polynucleotide sequence, respectively). The first polynucleotide sequence can be linked to a second polynucleotide sequence by a phosphodiester bond or a linker. The linker can be, e.g., a polynucleotide.

The terms “miRNA” or “miR” or “microRNA” are used interchangeably and refer to a microRNA molecule found in eukaryotes that is involved in RNA-based gene regulation. The term will be used to refer to the single-stranded RNA molecule processed from a precursor. Names of miRNAs and their sequences related to the present disclosure are provided herein. MicroRNAs recognize and bind to target mRNAs through imperfect base pairing leading to destabilization or translational inhibition of the target mRNA and thereby downregulate target gene expression. Conversely, targeting miRNAs via molecules comprising a miRNA binding site (generally a molecule comprising a sequence complementary to the seed region of the miRNA) can reduce or inhibit the miRNA-induced translational inhibition leading to an upregulation of the target gene.

The terms “mismatch” or “mismatches” refer to one or more nucleobases (whether contiguous or separate) in an oligomer nucleobase sequence that are not matched to a target pre-mRNA according to base pairing rules. While perfect complementarity is often desired, some embodiments can include one or more but preferably 6, 5, 4, 3, 2, or 1 mismatches with respect to the target pre-mRNA. Variations at any location within the oligomer are included. In certain embodiments, antisense oligomers of the disclosure include variations in nucleobase sequence near the termini, variations in the interior, and if present are typically within about 6, 5, 4, 3, 2, or 1 subunits of the 5′ and/or 3′ terminus. In certain embodiments, one, two, or three nucleobases can be removed and still provide on-target binding.

As used herein, the terms “modulate,” “modify,” and grammatical variants thereof, generally refer when applied to a specific concentration, level, expression, function or behavior, to the ability to alter, by increasing or decreasing, e.g., directly or indirectly promoting/stimulating/up-regulating or interfering with/inhibiting/down-regulating the specific concentration, level, expression, function or behavior, such as, e.g., to act as an antagonist or agonist. In some instances a modulator can increase and/or decrease a certain concentration, level, activity or function relative to a control, or relative to the average level of activity that would generally be expected or relative to a control level of activity.

“Nucleic acid,” “nucleic acid molecule,” “nucleotide sequence,” “polynucleotide,” and grammatical variants thereof are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The terms “pharmaceutically-acceptable carrier,” “pharmaceutically-acceptable excipient,” and grammatical variations thereof, encompass any of the agents approved by a regulatory agency of the U.S. Federal government or listed in the U.S. Pharmacopeia for use in animals, including humans, as well as any carrier or diluent that does not cause the production of undesirable physiological effects to a degree that prohibits administration of the composition to a subject and does not abrogate the biological activity and properties of the administered compound. Included are excipients and carriers that are useful in preparing a pharmaceutical composition and are generally safe, non-toxic, and desirable.

As used herein, the term “pharmaceutical composition” refers to one or more of the compounds described herein, such as, e.g., an EV, such as exosome of the present disclosure, mixed or intermingled with, or suspended in one or more other chemical components, such as pharmaceutically-acceptable carriers and excipients. One purpose of a pharmaceutical composition is to facilitate administration of preparations of EVs, e.g., exosomes, to a subject.

The term “plasmid” refers to an extra-chromosomal element often carrying a gene that is not part of the central metabolism of the cell, and usually in the form of circular double-stranded DNA molecules. Such elements can be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In some aspects of the present disclosure, the biologically active molecule attached to the EV, e.g., exosome, via a maleimide moiety is a polynucleotide, e.g., an antisense oligonucleotide. In particular aspects, the polynucleotide comprises an mRNA. In other aspect, the mRNA is a synthetic mRNA. In some aspects, the synthetic mRNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine). In some aspects of the present disclosure, the biologically active molecule is a polynucleotide.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art. The term “polypeptide,” as used herein, refers to proteins, polypeptides, and peptides of any size, structure, or function. Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to 50 amino acids long, e.g., about 5, 10, 15, 20, 25, 30, 35, 40, 45, or 50 amino acids long.

The terms “prevent,” “preventing,” and variants thereof as used herein, refer partially or completely delaying onset of an disease, disorder and/or condition; partially or completely delaying onset of one or more symptoms, features, or clinical manifestations of a particular disease, disorder, and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition. In some aspects, preventing an outcome is achieved through prophylactic treatment.

The terms “promoter” and “promoter sequence” herein are used interchangeably and refer to a DNA sequence capable of controlling the expression of a coding sequence or functional RNA. In general, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity.

The promoter sequence is typically bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase. In some embodiments, the nucleic acid molecule comprises a tissue specific promoter.

As used herein, “prophylactic” refers to a therapeutic or course of action used to prevent the onset of a disease or condition, or to prevent or delay a symptom associated with a disease or condition.

As used herein, a “prophylaxis” refers to a measure taken to maintain health and prevent or delay the onset of a bleeding episode, or to prevent or delay symptoms associated with a disease or condition.

As used herein, the term “gene regulatory region” or “regulatory region” refers to nucleotide sequences located upstream (5′ non-coding sequences), within, or downstream (3′ non-coding sequences) of a coding region, and which influence the transcription, RNA processing, stability, or translation of the associated coding region. Regulatory regions can include promoters, translation leader sequences, introns, polyadenylation recognition sequences, RNA processing sites, effector binding sites, or stem-loop structures. If a coding region is intended for expression in a eukaryotic cell, a polyadenylation signal and transcription termination sequence will usually be located 3′ to the coding sequence.

A polynucleotide which encodes a miRNA binding side of the present disclosure can include a promoter and/or other expression (e.g., transcription) control elements operably associated with one or more coding regions. In an operable association a coding region for a gene product is associated with one or more regulatory regions in such a way as to place expression of the gene product under the influence or control of the regulatory region(s). For example, a coding region and a promoter are “operably associated” if induction of promoter function results in the transcription of mRNA encoding the gene product encoded by the coding region, and if the nature of the linkage between the promoter and the coding region does not interfere with the ability of the promoter to direct the expression of the gene product or interfere with the ability of the DNA template to be transcribed. Other expression control elements, besides a promoter, for example enhancers, operators, repressors, and transcription termination signals, can also be operably associated with a coding region to direct gene product expression.

As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art. It is understood that percentage of similarity is contingent on the comparison scale used, i.e., whether the amino acids are compared, e.g., according to their evolutionary proximity, charge, volume, flexibility, polarity, hydrophobicity, aromaticity, isoelectric point, antigenicity, or combinations thereof.

The terms “subject,” “patient,” “individual,” and “host,” and variants thereof are used interchangeably herein and refer to any mammalian subject, including without limitation, humans, domestic animals (e.g., dogs, cats and the like), farm animals (e.g., cows, sheep, pigs, horses and the like), and laboratory animals (e.g., monkey, rats, mice, rabbits, guinea pigs and the like) for whom diagnosis, treatment, or therapy is desired, particularly humans. The methods described herein are applicable to both human therapy and veterinary applications.

As used herein, the phrase “subject in need thereof” includes subjects, such as mammalian subjects, that would benefit from administration of a nucleic acid molecule, or vector of the disclosure, e.g., to improve hemostasis.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

As used herein the term “therapeutically effective amount” is the amount of reagent or pharmaceutical compound comprising an EV or exosome of the present disclosure that is sufficient to a produce a desired therapeutic effect, pharmacologic and/or physiologic effect on a subject in need thereof. A therapeutically effective amount can be a “prophylactically effective amount” as prophylaxis can be considered therapy.

As used herein, “transcriptional control sequences” refer to DNA regulatory sequences, such as promoters, enhancers, terminators, and the like, that provide for the expression of a coding sequence in a host cell. A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit B-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

The terms “treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also include prophylaxis or prevention of a disease or condition or its symptoms thereof. In one aspect, the term “treating” or “treatment” means inducing an immune response in a subject against an antigen.

The term “upstream” refers to a nucleotide sequence that is located 5′ to a reference nucleotide sequence. In certain embodiments, upstream nucleotide sequences relate to sequences that are located on the 5′ side of a coding region or starting point of transcription. For example, most promoters are located upstream of the start site of transcription.

A “vector” refers to any vehicle for the cloning of and/or transfer of a nucleic acid into a host cell. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “vector” includes both viral and nonviral vehicles for introducing the nucleic acid into a cell in vitro, ex vivo or in vivo. A large number of vectors are known and used in the art including, for example, plasmids, modified eukaryotic viruses, or modified bacterial viruses. Insertion of a polynucleotide into a suitable vector can be accomplished by ligating the appropriate polynucleotide fragments into a chosen vector that has complementary cohesive termini.

Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), β-galactosidase (LacZ), β-glucuronidase (Gus), and the like. Selectable markers can also be considered to be reporters.

### II. Methods of Treatment and Use

The present disclosure also provides methods of treatment of a disease or a condition associated with increased level of a Nuclear receptor subfamily 4 group A member 2 (Nurr1) protein comprising the administration of a vector, e.g., an AAV vector, a polynucleotide, or a pharmaceutical composition of the present disclosure to a subject in need thereof. In some aspects, the present disclosure provides a method of treating a neurodegenerative disorder in a subject in need thereof, comprising administering to the subject a therapeutically effective amount of a vector, e.g., an AAV vector, a polynucleotide, or a pharmaceutical composition of the present disclosure.

The present disclosure provides that a miR204 inhibitor can reduce an expression of a Nurr1 protein in a subject in need thereof. Nurr1 protein is also known as Immediate-early response protein NOT, Orphan nuclear receptor NURR1, or Transcriptionally-inducible nuclear receptor. The gene names are known as NR4A2, NOT, NURR1, or TINUR. The amino acid sequence of a Nurr1 protein isoform 1 contains 598 amino acids. Its isoform 2 is missing amino acids 1-63. The sequence of isoform 1 (SEQ ID NO: 63) is shown below:

```
                10 20 30 40 50
MPCVQAQYGS SPQGASPASQ SYSYHSSGEY SSDFLTPEFV KFSMDLTNTE
                60 70 80 90 100
ITATTSLPSF STFMDNYSTG YDVKPPCLYQ MPLSGQQSSI KVEDIQMHNY
              110 120 130 140 150
QQHSHLPPQS EEMMPHSGSV YYKPSSPPTP TTPGFQVQHS PMWDDPGSLH
              160 170 180 190 200
NFHQNYVATT HMIEQRKTPV SRLSLFSFKQ SPPGTPVSSC QMRFDGPLHV
              210 220 230 240 250
PMNPEPAGSH HVVDGQTFAV PNPIRKPASM GFPGLQIGHA SQLLDTQVPS
              260 270 280 290 300
PPSRGSPSNE GLCAVCGDNA ACQHYGVRTC EGCKGFFKRT VQKNAKYVCL
              310 320 330 340 350
ANKNCPVDKR RRNRCQYCRF QKCLAVGMVK EVVRTDSLKG RRGRLPSKPK
              360 370 380 390 400
SPQEPSPPSP PVSLISALVR AHVDSNPAMT SLDYSRFQAN PDYQMSGDDT
              410 420 430 440 450
QHIQQFYDLL TGSMEIIRGW AEKIPGFADL PKADQDLLFE SAFLELFVLR
              460 470 480 490 500
LAYRSNPVEG KLIFCNGVVL HRLQCVRGFG EWIDSIVEFS SNLQNMNIDI
              510 520 530 540 550
SAFSCIAALA MVTERHGLKE PKRVEELQNK IVNCLKDHVT FNNGGLNRPN
              560 570 580 590
YLSKLLGKLP ELRTLCTQGL QRIFYLKLED LVPPPAIIDK LFLDTLPF
```

In some aspects, the miR204 inhibitor does not increase expression of an NMDA receptor. In other aspects, the miR204 inhibitor does not increase expression of a EphB2 protein.

In certain aspects, the miR204 inhibitor increases the expression of the Nurr1 protein after the administration or contact by at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, at least about 6 fold, at least about 6.5 fold, at least about 7 fold, at least about 7.5 fold, or at least about 8 fold compared to the expression prior to the administration or contact.

In some other aspects, the miR204 inhibitor treats a disease or condition associated with a decreased expression of the Nurr1 protein, but not with a decreased expression of an NMDA receptor and/or an EphB2 protein. In some aspects, the disease or condition is not associated with a decreased hippocampus function.

In certain aspects, the disease or condition is Alzheimer disease.

In other aspects, the disease or condition is Parkinson's disease, prion disease, motor neuron disease, Huntington's disease, spinocerebellar ataxia, spinal muscular atrophy, amyotrophic lateral sclerosis, or any combination thereof.

In some aspects, administering a vector, e.g., an AAV vector, a polynucleotide, or a pharmaceutical composition of the present disclosure improves one or more cognitive symptom in the subject, relative to the cognitive symptom in the subject prior to the administering. In some aspects, administering a vector, e.g., an AAV vector, a polynucleotide, or a pharmaceutical composition of the present disclosure improves mild cognitive impairment (MCI) in the subject. Experts classify mild cognitive impairment based on the thinking skills affected: MCI that primarily affects memory is known as “amnestic MCI.” With amnestic MCI, a person may start to forget important information that he or she would previously have recalled easily, such as appointments, conversations or recent events. MCI that affects thinking skills other than memory is known as “nonamnestic MCI.” Thinking skills that may be affected by nonamnestic MCI include the ability to make sound decisions, judge the time or sequence of steps needed to complete a complex task, or visual perception.

In some aspects, administering a vector, e.g., an AAV vector, a polynucleotide, or a pharmaceutical composition of the present disclosure reduces the occurrence or risk of occurrence of one or more symptoms of cognitive impairments in a subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to subjects not treated with at least a vector, e.g., an AAV vector, a polynucleotide, or a pharmaceutical composition of the present disclosure.

In some aspects, administering a vector, e.g., an AAV vector, a polynucleotide, or a pharmaceutical composition of the present disclosure reduces memory loss in the subject, relative to the memory loss in the subject prior to the administering. In some aspects, administering a vector, e.g., an AAV vector, a polynucleotide, or a pharmaceutical composition of the present disclosure reduces memory loss or the risk of occurrence of memory loss in a subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to subjects not treated with at least a vector, e.g., an AAV vector, a polynucleotide, or a pharmaceutical composition of the present disclosure.

In some aspects, administering a vector, e.g., an AAV vector, a polynucleotide, or a pharmaceutical composition of the present disclosure improves memory retention in the subject, relative to the memory retention in the subject prior to the administering. In some aspects, administering a vector, e.g., an AAV vector, a polynucleotide, or a pharmaceutical composition of the present disclosure improves and/or increases memory retention in a subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to subjects not treated with at least a vector, e.g., an AAV vector, a polynucleotide, or a pharmaceutical composition of the present disclosure.

In some aspects, administering a vector, e.g., an AAV vector, a polynucleotide, or a pharmaceutical composition of the present disclosure reduces an amyloid beta (Aβ) plaque load in the subject, relative to the amyloid beta (Aβ) plaque load in the subject prior to the administering. In some aspects, administering a vector, e.g., an AAV vector, a polynucleotide, or a pharmaceutical composition of the present disclosure reduces an amyloid beta plaque load, prevents or inhibits the development of an amyloid beta plaque load, delays the onset of the development of an amyloid beta plaque load, or lowers the risk of developing an amyloid beta plaque load in a subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to subjects not treated with at least a vector, e.g., an AAV vector, a polynucleotide, or a pharmaceutical composition of the present disclosure.

In some aspects, administering a vector, e.g., an AAV vector, a polynucleotide, or a pharmaceutical composition of the present disclosure increases dendritic spine density of a neuron in the subject, relative to the dendritic spine density of a neuron in the subject prior to the administering. In some aspects, administering a vector, e.g., an AAV vector, a polynucleotide, or a pharmaceutical composition of the present disclosure increases dendritic spine density of a neuron, decreases the loss of dendritic spines of a neuron, slows down the loss of dendritic spines of a neuron, prevents the loss of dendritic spines of a neuron, delays the onset of the loss of dendritic spines on a neuron, reduces the risk of loss of dendritic spines of a neuron, or a combination thereof in a subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to subjects not treated with at least a vector, e.g., an AAV vector, a polynucleotide, or a pharmaceutical composition of the present disclosure.

The present disclosure also provides a method of improving one or more cognitive symptoms of Alzheimer's disease in a subject in need thereof, comprising administering to the subject a vector, e.g., an AAV vector, a polynucleotide, or a pharmaceutical composition of the present disclosure. In some aspects, administering a vector, e.g., an AAV vector, a polynucleotide, or a pharmaceutical composition of the present disclosure reduces the occurrence or risk of occurrence of one or more cognitive symptoms of Alzheimer's disease in a subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to subjects not treated with at least a vector, e.g., an AAV vector, a polynucleotide, or a pharmaceutical composition of the present disclosure.

The present disclosure also provides a method of improving one or more cognitive symptoms of Parkinson disease in a subject in need thereof, comprising administering to the subject a vector, e.g., an AAV vector, a polynucleotide, or a pharmaceutical composition of the present disclosure. In some aspects, administering a vector, e.g., an AAV vector, a polynucleotide, or a pharmaceutical composition of the present disclosure improves one or more cognitive symptoms of Parkinson disease in a subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to subjects not treated with at least a vector, e.g., an AAV vector, a polynucleotide, or a pharmaceutical composition of the present disclosure.

The present disclosure also provides a method of improving one or more motor symptoms or non-motor symptoms of Parkinson disease in a subject in need thereof, comprising administering to the subject a vector, e.g., an AAV vector, a polynucleotide, or a pharmaceutical composition of the present disclosure. In some aspects, administering a vector, e.g., an AAV vector, a polynucleotide, or a pharmaceutical composition of the present disclosure improves one or more motor symptoms of Parkinson disease in a subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to subjects not treated with at least a vector, e.g., an AAV vector, a polynucleotide, or a pharmaceutical composition of the present disclosure. Motor symptoms considered cardinal in the diagnosis or Parkinson's disease are tremor, slowness of movement (bradykinesia), rigidity, and postural instability. Non-motor symptoms include autonomic dysfunction, neuropsychiatric problems (mood, cognition, behavior, or thought alterations), sensory alterations (especially altered sense of smell), and sleep difficulties. Alterations in the autonomic nervous system can lead to orthostatic hypotension (low blood pressure upon standing), oily skin and excessive sweating, urinary incontinence, and altered sexual function. Constipation and impaired stomach emptying (gastric dysmotility) can be severe enough to cause discomfort and even endanger health. Parkinson's disease can cause neuropsychiatric disturbances, which can range from mild to severe. The most common cognitive deficit in Parkinson's disease is executive dysfunction. Other cognitive difficulties include slowed cognitive processing speed, impaired recall and impaired perception and estimation of time. Visuospatial difficulties are also part of the disease. A person with Parkinson's disease has two to six times the risk of dementia compared to the general population. Impulse control disorders including pathological gambling, compulsive sexual behavior, binge eating, compulsive shopping and reckless generosity can be caused by medication, particularly orally active dopamine agonists. The most frequent mood difficulties are depression, apathy, and anxiety. Hallucinations or delusions occur in approximately 50% of people with Parkinson's disease over the course of the illness, and may herald the emergence of dementia.

The present disclosure also provides a method of improving synaptic function in a subject having a neurodegenerative disease, e.g., Parkinson's disease or Alzheimer's, comprising administering to the subject a vector, e.g., an AAV vector, a polynucleotide, or a pharmaceutical composition of the present disclosure. In some aspects, administering a vector, e.g., an AAV vector, a polynucleotide, or a pharmaceutical composition of the present disclosure improves synaptic function in a subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to subjects not treated with at least a vector, e.g., an AAV vector, a polynucleotide, or a pharmaceutical composition of the present disclosure.

The present disclosure also provides a method of preventing, delaying, or ameliorating the loss of synaptic function in a subject having a neurodegenerative disease, e.g., Parkinson's disease or Alzheimer's, comprising administering to the subject a vector, e.g., an AAV vector, a polynucleotide, or a pharmaceutical composition of the present disclosure. In some aspects, administering a vector, e.g., an AAV vector, a polynucleotide, or a pharmaceutical composition of the present disclosure prevents, delays, or ameliorates the loss of synaptic function in a subject having a neurodegenerative disease, e.g., Parkinson's disease or Alzheimer's by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to subjects not treated with at least a vector, e.g., an AAV vector, a polynucleotide, or a pharmaceutical composition of the present disclosure.

The present disclosure also provides a method of increasing dendritic spine density, delaying the decrease of dendritic spine density, ameliorating the decrease of dendritic spine density, stopping the decrease of dendritic spine density, preventing the decrease of dendritic spine density, maintaining dendritic spine density, in a subject having a neurodegenerative disease, e.g., Parkinson's disease or Alzheimer's, comprising administering to the subject a vector, e.g., an AAV vector, a polynucleotide, or a pharmaceutical composition of the present disclosure. In some aspects, administering a vector, e.g., an AAV vector, a polynucleotide, or a pharmaceutical composition of the present disclosure increases dendritic spine density, delays the decrease of dendritic spine density, ameliorates the decrease of dendritic spine density, stops the decrease of dendritic spine density, prevents the decrease of dendritic spine density, or maintains dendritic spine density in a subject having a neurodegenerative disease, e.g., Parkinson's disease or Alzheimer's by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to subjects not treated with at least a vector, e.g., an AAV vector, a polynucleotide, or a pharmaceutical composition of the present disclosure.

The present disclosure also provides a method of increasing dendritic spine density, delaying the decrease of dendritic spine density, ameliorating the decrease of dendritic spine density, stopping the decrease of dendritic spine density, preventing the decrease of dendritic spine density, maintaining dendritic spine density, in a subject having a neurodegenerative disease, e.g., Parkinson's disease or Alzheimer's, comprising administering to the subject a vector, e.g., an AAV vector, a polynucleotide, or a pharmaceutical composition of the present disclosure. In some aspects, administering a vector, e.g., an AAV vector, a polynucleotide, or a pharmaceutical composition of the present disclosure increases dendritic spine density, delays the decrease of dendritic spine density, ameliorates the decrease of dendritic spine density, stops the decrease of dendritic spine density, prevents the decrease of dendritic spine density, or maintains dendritic spine density in a subject having a neurodegenerative disease, e.g., Parkinson's disease or Alzheimer's by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to subjects not treated with at least a vector, e.g., an AAV vector, a polynucleotide, or a pharmaceutical composition of the present disclosure.

Amyloid beta (Aβ) plaque is known to cause neuronal changes, e.g., aberrations in synapse composition, synapse shape, synapse density, loss of synaptic conductivity, changes in dendrite diameter, changes in dendrite length, changes in spine density, changes in spine area, changes in spine length, or changes in spine head diameter. Accordingly, the present disclosure also provides methods of treating, preventing, decreasing, delaying the onset, stopping further progression, ameliorating the aforementioned changes in a subject having a neurodegenerative disease characterized by the deposition of AP plaque, e.g., Alzheimer's disease, comprising administering to the subject a vector, e.g., an AAV vector, a polynucleotide, or a pharmaceutical composition of the present disclosure. In some aspects, administering a vector, e.g., an AAV vector, a polynucleotide, or a pharmaceutical composition of the present disclosure treats, prevents, decreases, delays the onset, stops further progression, or ameliorates the aforementioned changes in a subject having a neurodegenerative disease characterized by the deposition of AP plaque, e.g., Alzheimer's disease, by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to subjects not treated with at least a vector, e.g., an AAV vector, a polynucleotide, or a pharmaceutical composition of the present disclosure.

It is known in the art that Aβ plaque is also present in some variants of Lewy body dementia, inclusion body myositis, cerebral amyloid angiopathy, and Down syndrome (the gene for the amyloid precursor protein is located on chromosome 21, and accordingly people with Down syndrome have a very high incidence of Alzheimer's disease).

In some aspects of the methods disclosed herein, the Alzheimer's disease is pre-dementia Alzheimer's disease, early Alzheimer's disease, moderate Alzheimer's disease, advanced Alzheimer's disease, early onset familial Alzheimer's disease, inflammatory Alzheimer's disease, non-inflammatory Alzheimer's disease, cortical Alzheimer's disease, early-onset Alzheimer's disease, or late-onset Alzheimer's disease.

In some aspects, the vector, e.g., an AAV vector, polynucleotide, or pharmaceutical composition of the present disclosure is administered via intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

In some aspects, the vectors, e.g., an AAV vector, polynucleotides, or pharmaceutical compositions of the present disclosure can be used concurrently with other medicaments or treatment suitable for the treatment of the diseases and conditions disclosed herein.

### III. Vectors Useful for the Disclosure

The present disclosure is directed to vectors encoding at least an miR-204 binding site. The term “miR-204 binding site” can also be used interchangeably with miR-204 antimir, miR-204 antagomir, or anti-miR204 oligonucleotide. In some aspects, a vector of the present disclosure, e.g., an AVV vector, comprises one or more regulatory elements (e.g., a promoter) and one RNA expression region located, e.g., downstream from a regulatory element (e.g., a promoter), wherein the RNA expression region comprises a nucleotide sequence encoding an RNA comprising at least one miR-204 binding site. In a specific aspect, the RNA expression region does not encode a protein. In some aspects, the vector is double stranded. In other aspects, the vector is single stranded.

In some aspects, the miR-204 binding site is a single-stranded polynucleotide sequence that is complementary to a sequence of a mature miR-204-5p (SEQ ID NO:1) or miR-204-3p (SEQ ID NO:5), which functions as an inhibitor of miR-204-5p or miR-204-3p (miR-204), respectively. Non-limiting examples of various aspects are shown in the present disclosure.

The miR-204 hairpin precursor can generate both miR-204-5p and miR-204-3p. In the context of the present disclosure “miR-204” encompasses both miR-204-5p and miR-204-3p unless specified otherwise. The human mature miR-204-5p has the sequence 5′-uucccuuugucauccuaugccu-3′ (SEQ ID NO:5; miRBase Acc. No. MIMAT0000265). The 5′ terminal subsequence of miR-204-5p 5′-uucccuu-3′ (SEQ ID NO:25) is the seed sequence. The human mature miR-204-3p has the sequence 5′-gcugggaaggcaaagggacgu-3′ (SEQ ID NO:5; miRBase Acc. No. MIMAT0022693). The 5′ terminal subsequence of miR-204-3p 5′-gcuggga-3′ (SEQ ID NO:26) is the seed sequence.

The seed region of a miRNA forms a tight duplex with the target mRNA. Most miRNAs imperfectly base-pair with the 3′ untranslated region (UTR) of target mRNAs, and the 5′ proximal “seed” region of miRNAs provides most of the pairing specificity. Without being bound to any theory, it is believed that the first nine miRNA nucleotides (encompassing the seed sequence) provide greater specificity whereas the miRNA ribonucleotides 3′ of this region allow for lower sequence specificity and thus tolerate a higher degree of mismatched base pairing, with positions 2-7 being the most important. Accordingly, in specific aspects of the present disclosure, the miR-204 binding site comprises a subsequence that is fully complementary (i.e., 100% complementary) over the entire length of the seed sequence of miR-204.

miRNA sequences and miRNA binding sequences that can be used in the context of the disclosure include, but are not limited to, all or a portion of those sequences in the sequence listing provided herein, as well as the miRNA precursor sequence, or complement of one or more of these miRNAs. Any aspects of the disclosure involving specific miRNAs or miRNA binding sites by name is contemplated also to cover miRNAs or complementary sequences thereof whose sequences are at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to the mature sequence of the specified miRNA sequence or complementary sequence thereof.

In some aspects, miRNA binding sequences of the present disclosure can include additional nucleotides at the 5′, 3′, or both 5′ and 3′ ends of those sequences in the sequence listing provided herein, as long as the modified sequence is still capable of specifically binding to miR-204. In some aspects, miRNA binding sequences of the present disclosure can differ in at least 1, 2, 3, 4, 5, 6, 7, 8, 9, 10 or more nucleotides with respect to those sequence in the sequence listing provided, as long as the modified sequence is still capable of specifically binding to miR-204.

It is also specifically contemplated that any methods and compositions discussed herein with respect to miRNA binding molecules or miRNA may be implemented with respect to synthetic miRNAs binding molecules. It is also understood that the disclosures related to RNA sequences in the present disclosure are equally applicable to corresponding DNA sequences.

In some aspects, the As noted elsewhere herein, the RNA expression region in a vector of the present disclosure does not encode a protein, e.g., the vector does not encode a protein that is heterologous to the vector. However, in other aspects, the RNA expression region of the vector, in addition to expressing miR-204 binding sites, can also express polynucleotides other than the miR-204 binding sites, and/or one or more additional RNAs.

The miR-204 binding site or sites expressed by the vector of the present disclosure can bind to endogenous miR-204, regulating, e.g., expression of one or more endogenous polypeptides (e.g., EphB2 or SIRT1), which in turns treats or ameliorate the symptoms of a neurodegenerative disease, e.g., Alzheimer's disease or Parkinson's disease.

In some aspects, the vector of the present disclosure increases expression of one or more endogenous polypeptide, e.g., SIRT1. SIRT1, also known as NAD-dependent deacetylase sirtuin-1 (Uniprot Q96EB6), is a protein that in human is encoded by the SIRT1 gene. mRNAs encoding SIRT1 known in the art include, e.g., RefSeq sequences NM_001142498, NM_001314049, and NM_012238.

In some aspects, the at least one miR-204 binding site expressed by the vector of the present disclosure hybridizes to mature miR-204-5p (SEQ ID NO:1) or a subsequence thereof. In some aspects, the miR-204-5p subsequence comprises the seed sequence.

In some aspects, the at least one miR-204 binding site expressed by the vector of the present disclosure is fully complementary to miR-204-5p. In some aspects, the at least one miR-204 binding site expressed by the vector of the present disclosure comprises the nucleic acid sequence set forth in SEQ ID NO:2. In some aspects, the sequence encoding the at least one miR-204 binding site in the vector comprises the sequence set forth in SEQ ID NO:3.

In some aspects, the at least one miR-204 binding site expressed by the vector of the present disclosure is complementary to miR-204-5p except for 1, 2, 3, 4, 5, 6, 7, 8, 9, or 10 mismatches. In some aspects, the none of the mismatches are in the subsequence complementary to the miR-204-5p seed sequence. In some aspects, the at least one miR-204 binding site comprises a sequence fully complementary to the seed sequence plus 1, 2, 3, 4, 5, 6, 7, 8, 9, 10, 11, 12, 13, 14 or 15 nucleotides extending in the 5′ direction beyond the region of complementary to miR-204-5p seed sequence.

In some aspects, the at least one miR-204 binding site expressed by the vector of the present disclosure hybridizes to mature miR-204-3p (SEQ ID NO:5) or a subsequence thereof. In some aspects, the miR-204-3p subsequence comprises the seed sequence.

In some aspects, the at least one miR-204 binding site expressed by the vector of the present disclosure is fully complementary to miR-204-3p. In some aspects, the at least one miR-204 binding site comprises the nucleic acid sequence set forth in SEQ ID NO:6. In some aspects, the sequence encoding the at least one miR-204 binding site in the vector comprises the sequence set forth in SEQ ID NO:7.

In some aspects, the at least one miR-204 binding site expressed by the vector of the present disclosure is complementary to miR-204-3p except for 1, 2, 3, 4, 5, 6, 7, 8, 9, or 10 mismatches. In some aspects, the none of the mismatches are in the subsequence complementary to the miR-204-3p seed sequence. In some aspects, the at least one miR-204 binding site comprises a sequence fully complementary to the seed sequence plus 1, 2, 3, 4, 5, 6, 7, 8, 9, 10, 11, 12, 13, 14 or 15 nucleotides extending in the 5′ direction beyond the region of complementary to miR-204-3p seed sequence.

In some aspects, the RNA expressed by the vector of the present disclosure comprises at least two miR-204 binding sites. In some aspects, the RNA expressed by the vector of the present disclosure comprises two miR-204 binding sites, three miR-204 binding sites, four miR-204 binding sites, five miR-204 binding sites, or six miR-204 binding sites. In some aspects, the RNA expressed by the vector of the present disclosure comprises two miR-204 binding sites. In some aspects, all the miR-204 binding sites are identical. In some aspects, all the miR-204 binding sites are different. In some aspects, at least one of the miR-204 binding sites is different. In some aspects, all the miR-204 binding sites are miR-204-5p binding sites. In other aspects, all the miR-204 binding sites are miR-205-3p binding sites.

In some aspects, the vector of the present disclosure is a virus, a plasmid, or a phagemid. In some aspects, the virus is selected from the group consisting of an adeno-associated virus (AAV), a retrovirus, a lentivirus, an adenovirus, an SV40-type virus, a polyomavirus, an Epstein-Barr virus, a papilloma viruses, a herpes virus, a vaccinia virus, a polio virus, and an RNA virus.

Any AAV vector known in the art can be used in the methods disclosed herein. The AAV vector can comprise a known vector or can comprise a variant, fragment, or fusion thereof. In some embodiments, the AAV vector is selected from the group consisting of AAV type 1 (AAV1), AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, and any combination thereof.

In some embodiments, the AAV vector is derived from an AAV vector selected from the group consisting of AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, and any combination thereof.

In some embodiments, the AAV vector is a chimeric vector derived from at least two AAV vectors selected from the group consisting of AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, and any combination thereof.

In certain embodiments, the AAV vector comprises regions of at least two different AAV vectors known in the art.

In some embodiments, the AAV vector comprises an inverted terminal repeat from a first AAV (e.g., AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, or any derivative thereof) and a second inverted terminal repeat from a second AAV (e.g., AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, or any derivative thereof).

In some aspects, the AVV vector comprises a portion of an AAV vector selected from the group consisting of AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, and any combination thereof. In some aspects, the AAV vector comprises AAV2.

In some aspects, the AVV vector comprises a splice acceptor site. In some aspects, the AVV vector comprises a promoter. Any promoter known in the art can be used in the AAV vector of the present disclosure. In some aspects, the promoter is an RNA Pol III promoter. In some aspects, the RNA Pol III promoter is selected from the group consisting of the U6 promoter, the H1 promoter, the 7SK promoter, the 5S promoter, the adenovirus 2 (Ad2) VAI promoter, and any combination thereof. In some aspects, the promoter is a cytomegalovirus immediate-early gene (CMV) promoter, an EF1a promoter, an SV40 promoter, a PGK1 promoter, a Ubc promoter, a human beta actin promoter, a CAG promoter, a TRE promoter, a UAS promoter, a Ac5 promoter, a polyhedrin promoter, a CaMKIIa promoter, a GAL1 promoter, a GAL10 promoter, a TEF promoter, a GDS promoter, a ADH1 promoter, a CaMV35S promoter, or a Ubi promoter. In a specific aspect, the promoter comprises the U6 promoter.

In some aspects, the AAV vector comprises a constitutively active promoter (constitutive promoter). In some aspects, the constitutive promoter is selected from the group consisting of hypoxanthine phosphoribosyl transferase (HPRT), adenosine deaminase, pyruvate kinase, beta-actin promoter, cytomegalovirus (CMV), simian virus (e.g., SV40), papilloma virus, adenovirus, human immunodeficiency virus (HIV), Rous sarcoma virus, a retrovirus long terminal repeat (LTR), Murine stem cell virus (MSCV) and the thymidine kinase promoter of herpes simplex virus.

In some aspects, the promoter is an inducible promoter. In some aspects, the inducible promoter is a tissue specific promoter. In certain aspects, the tissue specific promoter drives transcription of the coding region of the AVV vector in a neuron, a glial cell, or in both a neuron and a glial cell.

In some aspects, the AVV vector comprises one or more enhancers. In some aspects, the one or more enhancer are present in the AAV alone or together with a promoter disclosed herein. In some embodiments, the AAV vector comprises a 3′UTR poly(A) tail sequence. In some embodiments, the 3′UTR poly(A) tail sequence is selected from the group consisting of bGH poly(A), actin poly(A), hemoglobin poly(A), and any combination thereof. In some embodiments, the 3′UTR poly(A) tail sequence comprises bGH poly(A).

### IV. Polynucleotides Useful for the Present Disclosure

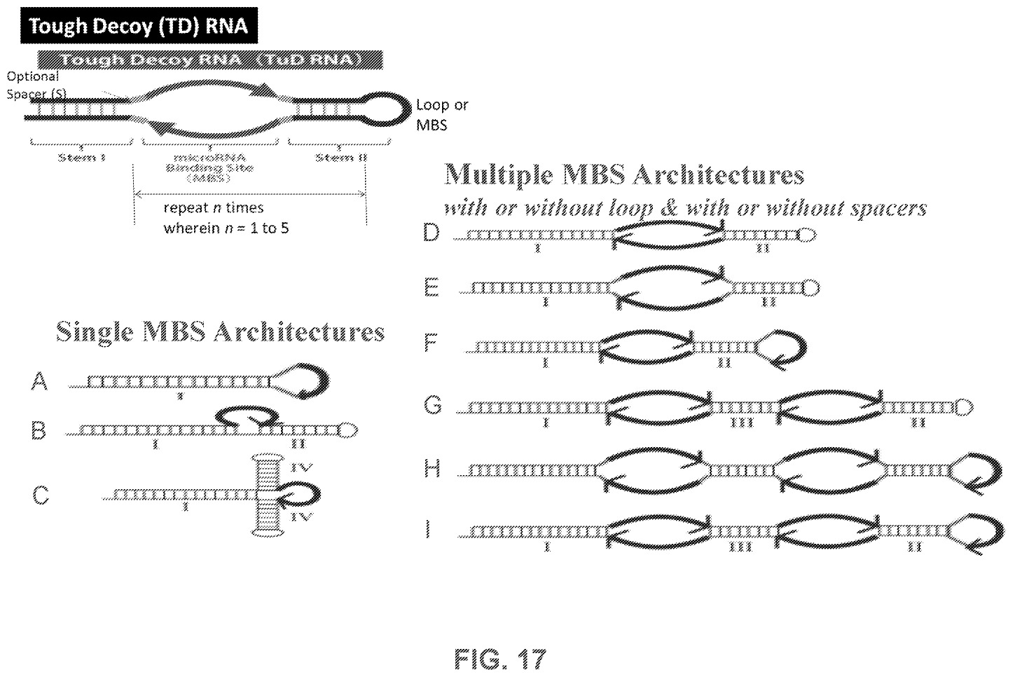

In some specific aspects, the vector of the present disclosure expresses an RNA comprising the nucleic acid sequence set forth in SEQ ID NO: 23. The architecture of such RNA is presented generally in the top left schematic representation in FIG. 17. The topology of the sequence of SEQ ID NO:23 is also presented in FIG. 17, schematic representation E.

In some aspects, the RNA of the present disclosure is a “TD RNA,” i.e., it is a polynucleotide that has a “Tough Decoy” (TD) topology as exemplified, for example, in the TD exemplary topologies presented in FIG. 17. The top left diagram in FIG. 17 shows the modular structure of a TD, showing in particular the location of MB S (microRNA binding sites), stems, and optional spacers. In the context of the present disclosure, the designations Stem I and Stem 1, and variants thereof (e.g., Stem II and Stem 2, Stem III and Stem 3) are interchangeable.

As used herein, the term “Tough Decoy,” abbreviated as “TD,” refers to a stabilized stem-loop RNA molecule with at least one microRNA binding domain (MBD). The TDs disclosed herein are artificial strands of RNA, produced either via vector-driven expression or via chemical or enzymatic in vitro synthesis, with miRNA-binding domains that are capable of sequestering a target miRNA into stable complexes through complementary base pairing, disabling a particular RNA interference pathway. Thus, while miRNAs act as repressors, TDs act as double-repressors such that the presence of the TDs increases protein output. In some aspects, the TDs of the present disclosure are incorporated into viral plasmids containing a mammalian promoter, e.g., a U6 promoter, to drive expression of the TD in vivo upon transfection into mammalian cells. Thus, in some aspects, the RNA comprising at least one miR-204 binding site disclosed herein, e.g., a TD, is expressed by a polynucleotide in a vector of the present disclosure, i.e., an AAV vector.

As used herein, the terms “expressed,” “expression,” and grammatical variants refer, when applied to a polynucleotide in a vector of the present disclosure, to the transcription of multiple copies of an RNA comprising at least one miR-204 binding site disclosed herein, e.g., a TD.

In some aspects, the TD RNA comprises a dual stranded first stem region (Stem 1), a dual stranded second stem region (Stem 2), two single stranded miR-204 binding sites (microRNA-204 binding site 1 and microRNA-204 binding site 2), and a Loop region. In some aspects the TD RNA has an organization corresponding to schema I:

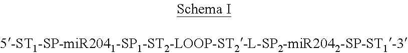

wherein
ST₁ and ST₁′ are complementary stem 1 sequences;
ST₂ and ST₂′ are complementary stem 2 sequences;
miR204₁ and miR204₂ are miR-204 binding sites (MBS);
LOOP is a loop sequence;
SP are optional spacer sequences; and
L is an optional linker sequence.

In some aspects, additional linker sequences can be present between the other elements of schema I, e.g., between ST₂′ and SP-miR204₂ to improve the stability of the TD construct.

In some aspects, ST₁ and ST₁′ comprise, consist, or consist essentially of the sequence 5′-gacggcgctaggatcatc-3′ (SEQ ID NO: 16) and 5′gatgatcctagctccgtc3′ (SEQ ID NO: 18), respectively. In some aspects, ST₁ and ST₁′ are fully complementary.

In some aspects, ST₂ and ST₂′ comprise, consist, or consist essentially of the sequence 5′-gtattctg-3′ (SEQ ID NO:10) and 5′-cagaatac-3′ (SEQ ID NO:12), respectively. In some aspects, ST₂ and ST₂′ are fully complementary.

In some aspects, the LOOP sequence comprises, consists, or consists essentially of the sequence 5′-gtca-3′ (SEQ ID NO: 14).

In some aspects, SP₁ comprises, consists, or consists essentially of the sequence 5′-acc-3′. In some aspects, SP₂ comprises, consists, or consists essentially of the sequence 5′-acc-3′. In some aspects, SP₁ and SP₂ are fully complementary.

In some aspects, L is a linker sequence comprising, consisting, or consisting essentially of the sequence 5′-aacaatac-3′.

In some aspects, both miR204₁ and miR204₂ are miR-204-5p binding sites. In some aspects, both miR204₁ and miR204₂ are miR-204-3p binding sites. In some aspects, miR204₁ is a miR-204-5p binding site, and miR204₂ is a miR-204-3p binding site. In some aspects, miR204₁ is a miR-204-3p binding site, and miR204₂ is a miR-204-5p binding site. In some aspects, miR204₁ and miR204₂ identical. In some aspects, and miR204₁ and miR204₂ are different. In some aspects where more than two miR-204ₓ binding sites are present, they can be either miRNA-204-5p binding sites or miRNA-204-30 binding sites, which in turn can be identical or different.

In some aspects, miR204₁ comprises the 22-mer nucleic acid sequence set forth in SEQ ID NO:2 (5′ aggcauaggaugacaaagggaa3′). In some aspects, miR204₁ consists of the 22-mer nucleic acid sequence set forth in SEQ ID NO:2 (5′ aggcauaggaugacaaagggaa3′). In some aspects, miR204₁ comprises 7, 8, 10, 11, 12, 13, 14, 15, 16, 17, 18, 19, 20, 21, or 22 continuous nucleotides from SEQ ID NO:2. In some aspects, miR204₁ consists of 7, 8, 9, 10, 11, 12, 13, 14, 15, 16, 17, 18, 19, 20, 21, or 22 continuous nucleotides from SEQ ID NO:2.

In some aspects, miR204₁ comprises 7, 8, 9, 10, 11, 12, 13, 14, 15, 16, 17, 18, 19, 20, 21, or 22 continuous nucleotides from SEQ ID NO:2, and at least 1, 2, 3, 4 or 5 additional 5′ terminal nucleotides. In some aspects, miR204₁ comprises 7, 8, 9, 10, 11, 12, 13, 14, 15, 16, 17, 18, 19, 20, 21, or 22 continuous nucleotides from SEQ ID NO:2, and at least 1, 2, 3, 4 or 5 additional 3′ terminal nucleotides. In some aspects, miR204₁ comprises 7, 8, 9, 10, 11, 12, 13, 14, 15, 16, 17, 18, 19, 20, 21, or 22 continuous nucleotides from SEQ ID NO:2, at least 1, 2, 3, 4 or 5 additional 5′ terminal nucleotides, and at least 1, 2, 3, 4 or 5 additional 3′ terminal nucleotides.

In some aspects, miR204₁ consists of 7, 8, 9, 10, 11, 12, 13, 14, 15, 16, 17, 18, 19, 20, 21, or 22 continuous nucleotides from SEQ ID NO:2, and at least 1, 2, 3, 4 or 5 additional 5′ terminal nucleotides. In some aspects, miR204₁ consists of 7, 8, 9, 10, 11, 12, 13, 14, 15, 16, 17, 18, 19, 20, 21, or 22 continuous nucleotides from SEQ ID NO:2, and at least 1, 2, 3, 4 or 5 additional 3′ terminal nucleotides. In some aspects, miR204₁ consists of 7, 8, 9, 10, 11, 12, 13, 14, 15, 16, 17, 18, 19, 20, 21, or 22 continuous nucleotides from SEQ ID NO:2, at least 1, 2, 3, 4 or 5 additional 5′ terminal nucleotides, and at least 1, 2, 3, 4 or 5 additional 3′ terminal nucleotides.

In some aspects, miR204₁ or a subsequence thereof differs from the nucleic acid sequence set forth in SEQ ID NO:2 (5′ aggcauaggaugacaaagggaa3′) or a subsequence thereof by 1, 2, 3, 4, 5, 6, 7, 8, 9, 10, 11, 12, 13, 14, or 15 nucleotides, wherein miR204₁ can still specifically bind to and inhibit the target microRNA.

In some aspects, miR204₂ comprises the 21-mer nucleic acid sequence set forth in SEQ ID NO:6 (5 acgucccuuugccuucccagc3′). In some aspects, miR204₂ consists of the 21-mer nucleic acid sequence set forth in SEQ ID NO:6 (5 acgucccuuugccuucccagc3′).

In some aspects, miR204₂ comprises 7, 8, 9, 10, 11, 12, 13, 14, 15, 16, 17, 18, 19, 20 or 21 continuous nucleotides from SEQ ID NO:6. In some aspects, miR204₂ consists of 7, 8, 9, 10, 11, 12, 13, 14, 15, 16, 17, 18, 19, 20 or 21 continuous nucleotides from SEQ ID NO:6.

In some aspects, miR204₂ comprises 7, 8, 9, 10, 11, 12, 13, 14, 15, 16, 17, 18, 19, 20, 21, or 22 continuous nucleotides from SEQ ID NO:6, and at least 1, 2, 3, 4 or 5 additional 5′ terminal nucleotides. In some aspects, miR204₂ comprises 7, 8, 9, 10, 11, 12, 13, 14, 15, 16, 17, 18, 19, 20, 21, or 22 continuous nucleotides from SEQ ID NO:6, and at least 1, 2, 3, 4 or 5 additional 3′ terminal nucleotides. In some aspects, miR204₂ comprises 7, 8, 9, 10, 11, 12, 13, 14, 15, 16, 17, 18, 19, 20, 21, or 22 continuous nucleotides from SEQ ID NO:6, at least 1, 2, 3, 4 or 5 additional 5′ terminal nucleotides, and at least 1, 2, 3, 4 or 5 additional 3′ terminal nucleotides.

In some aspects, miR204₂ consists of 7, 8, 9, 10, 11, 12, 13, 14, 15, 16, 17, 18, 19, 20, 21, or 22 continuous nucleotides from SEQ ID NO:6, and at least 1, 2, 3, 4 or 5 additional 5′ terminal nucleotides. In some aspects, miR204₂ consists of 7, 8, 9, 10, 11, 12, 13, 14, 15, 16, 17, 18, 19, 20, 21, or 22 continuous nucleotides from SEQ ID NO:6, and at least 1, 2, 3, 4 or 5 additional 3′ terminal nucleotides. In some aspects, miR204₂ consists of 7, 8, 9, 10, 11, 12, 13, 14, 15, 16, 17, 18, 19, 20, 21, or 22 continuous nucleotides from SEQ ID NO:6, at least 1, 2, 3, 4 or 5 additional 5′ terminal nucleotides, and at least 1, 2, 3, 4 or 5 additional 3′ terminal nucleotides.

In some aspects, miR204₂ or a subsequence thereof differs from the nucleic acid sequence set forth in SEQ ID NO:6 (5′ acgucccuuugccuucccagc3′) or a subsequence thereof by 1, 2, 3, 4, 5, 6, 7, 8, 9, 10, 11, 12, 13, 14, or 15 nucleotides, wherein miR204₂ can still specifically bind to and inhibit the target microRNA.

In some aspects, miR204₁ is a 7-mer, 8-mer, 9-mer or 10-mer comprising a subsequence of SEQ ID NO:2 fully complementary to the seed region of miR204-5p. Accordingly, in some aspects, miR204₁ consists of the sequence 5′-aagggaa-3′ plus 0, 1, 2, or 3 additional 5′ and/or 3′ nucleotides, wherein miR204₁ can still specifically bind to and inhibit the target microRNA.

In some aspects, miR204₂ is a 7-mer, 8-mer, 9-mer or 10-mer comprising a subsequence of SEQ ID NO:2 fully complementary to the seed region of miR204-5p. Accordingly, in some aspects, miR204₂ consists of the sequence 5′-ucccagc-3′ plus 0, 1, 2, or 3 additional 5′ and/or 3′ nucleotides, wherein miR204₂ can still specifically bind to and inhibit the target microRNA.

In some aspects, the TD comprises 1, 2, 3, 4, 5, or 6 microRNA binding sites (MBS). Exemplary topologies comprising one MBS are depicted, e.g., in architectures A, B, and C in FIG. 17. In some aspects, the MBS in architectures A, B, and C, or variants thereof comprising optional spacers, can be any miR204₁ or miR204₂ disclosed above. In some aspects, the TD comprises two MBS, as shown, e.g., in exemplary architectures D and E, wherein each MBS C can be any miR204₁ or miR204₂ disclosed above. In some aspects, the TD comprises three MBS, as shown, e.g., in exemplary architecture F in FIG. 17, or a variant thereof comprising optional spacers, wherein each MBS can be any miR204₁ or miR204₂ disclosed above. In some aspects, the TD comprises four MBS, as shown, e.g., in exemplary architecture G in FIG. 17, or a variant thereof comprising optional spacers, wherein each MBS can be any miR204₁ or miR204₂ disclosed above.

In some aspects, the TD comprises five MBS, as shown, e.g., in exemplary architectures H and I in FIG. 17 wherein each MBS can be any miR204₁ or miR204₂ disclosed above.

In some aspects, the vector of the present disclosure can express a single type of TD. In other aspects, the vector of the present disclosure can express more than one type TD, e.g., (i) TD with different architectures targeting the same miRNA (e.g., miR204-5p or miR204-3p); (ii) TD with different architectures targeting different miRNAs (e.g., miR204-5p or miR204-3p); (iii) TD with the same architecture targeting the same miRNA (e.g., miR204-5p or miR204-3p); or, (iv) combinations thereof. Similarly, compositions of the present disclosure comprising TD obtained by chemical or enzymatic in vitro synthesis can comprise (i) TD with different architectures targeting the same miRNA (e.g., miR204-5p or miR204-3p); (ii) TD with different architectures targeting different miRNAs (e.g., miR204-5p or miR204-3p); (iii) TD with the same architecture targeting the same miRNA (e.g., miR204-5p or miR204-3p); or, (iv) combinations thereof.

Non-limiting examples of the polynucleotides for the present disclosure are shown below:

| Subtypes | Sequence |
|---|---|
| RNA complex_204- | AACTCGAGGTTCGATACAGGGGCATCAAGAGGCATAGGATGACAAAGGGAAGAATTTGGAAC |
| 5p_subtype1 | GTCAGTTCCAAAAAGAAGAATAGAAGGCATAGGATGACAAAGGGAAGAAGATGCCCCTGTAT |
| (SEQ ID NO: 27) | CGAACTTTTTTGGAACTCGAGAA |
| RNA complex_204- | AACTCGAGTGCGCGCTTTGTGGATGTAAGAGGCATAGGATGACAAAGGGAAGAAACCCCAGT |
| 5p_subtype2 | GTCAACTGGGGTAAGAAGAATAGAAGGCATAGGATGACAAAGGGAAGAAACATCCACAAAGC |
| (SEQ ID NO: 28) | GCGCATTTTTTGGAACTCGAGAA |
| RNA complex_204- | AACTCGAGAATAACTAGACACAATCGAAGAGGCATAGGATGACAAAGGGAAGAAACCTTCTG |
| 5p_subtype3 | GUCACAGAAGGTAAGAAGAATAGAAGGCATAGGATGACAAAGGGAAGAACGATTGTGTCTAG |
| (SEQ ID NO: 29) | TTATTTTTTTTGGAACTCGAGAA |
| RNA complex_204- | AACTCGAGGTTTTTATATGCCAACACAAGAGGCATAGGATGACAAAGGGAAGAATACCCGAA |
| 5p_subtype4 | GTCATTCGGGTAAAGAAGAATAGAAGGCATAGGATGACAAAGGGAAGAAGTGTTGGCATATA |
| (SEQ ID NO: 30) | AAAACTTTTTTGGAACTCGAGAA |
| RNA complex_204- | AAGGATCCCCTTGGGCTTGTAGCCTAAAGAGGCATAGGATGACAAAGGGAAGAAGTTGAGCG |
| 5p_subtype5 | GTCACGCTCAACAAGAAGAATAGAAGGCATAGGATGACAAAGGGAAGAATAGGCTACAAGCC |
| (SEQ ID NO: 31) | CAAGGTTTTTTGGAACTCGAGAA |
| RNA complex_204- | AAGGATCCTGTGGATTTTCCAAAAGGAAGAGGCATAGGATGACAAAGGGAAGAAAAGATCGT |
| 5p_subtype6 | GTCAACGATCTTAAGAAGAATAGAAGGCATAGGATGACAAAGGGAAGAACCTTTTGGAAAAT |
| (SEQ ID NO: 32) | CCACATTTTTTGGAACTCGAGAA |
| RNA complex_204- | AAGGATCCAAGTCAGCCTTATTGGACAAGAGGCATAGGATGACAAAGGGAAGAAAGCAATAG |
| 5p_subtype7 | GTCACTATTGCTAAGAAGAATAGAAGGCATAGGATGACAAAGGGAAGAAGTCCAATAAGGCT |
| (SEQ ID NO: 33) | GACTTTTTTTTGGAACTCGAGAA |
| RNA complex_204- | AAGGATCCTACGACCGAGTTCGTAGTAAGAGGCATAGGATGACAAAGGGAAGAACAGTGAAA |
| 5p_subtype8 | GTCATTTCACTGAAGAAGAATAGAAGGCATAGGATGACAAAGGGAAGAAACTACGAACTCGG |
| (SEQ ID NO: 34) | TCGTATTTTTTGGAACTCGAGAA |
| RNA complex_204- | AAGGATCCCCCGGCGAAATACTGCGAAAGAGGCATAGGATGACAAAGGGAAGAATAGCATTC |
| 5p_subtype9 | GTCAGAATGCTAAAGAAGAATAGAAGGCATAGGATGACAAAGGGAAGAATCGCAGTATTTCG |
| (SEQ ID NO: 35) | CCGGGTTTTTTGGAACTCGAGAA |
| RNA complex_204- | AAGGATCCTGGTGGGGACACTGAGCTAAGAGGCATAGGATGACAAAGGGAAGAAGTCTCGCA |
| 5p_subtype10 | GTCATGCGAGACAAGAAGAATAGAAGGCATAGGATGACAAAGGGAAGAAAGCTCAGTGTCCC |
| (SEQ ID NO: 36) | CACCATTTTTTGGAACTCGAGAA |
| RNA complex_204- | AAGGATCCTAAACAGATCAACACGCTAAGAGGCATAGGATGACAAAGGGAAGAAAGTTGGGC |
| 5p_subtype11 | GTCAGCCCAACTAAGAAGAATAGAAGGCATAGGATGACAAAGGGAAGAAAGCGTGTTGATCT |
| (SEQ ID NO: 37) | GTTTATTTTTTGGAACTCGAGAA |
| RNA complex_204- | AAGGATCCCGTCCTACTTAAGAGAGTAAGAGGCATAGGATGACAAAGGGAAGAAGTCTATAC |
| 5p_subtype12 | GUCAGTATAGACAAGAAGAATAGAAGGCATAGGATGACAAAGGGAAGAAACTCTCTTAAGTA |
| (SEQ ID NO: 38) | GGACGTTTTTTGGAACTCGAGAA |
| RNA complex_204- | AAGGATCCTGTGTTCCGCGGAGATCAAAGAGGCATAGGATGACAAAGGGAAGAAACAAAAAC |
| 5p_subtype13 | GTCAGTTTTTGTAAGAAGAATAGAAGGCATAGGATGACAAAGGGAAGAATGATCTCCGCGGA |
| (SEQ ID NO: 39) | ACACATTTTTTGGAACTCGAGAA |
| RNA complex_204- | AAGGATCCTCCTGATGTAAGCTTACAAAGAGGCATAGGATGACAAAGGGAAGAAAATTATTC |
| 5p_subtype14 | GTCAGAATAATTAAGAAGAATAGAAGGCATAGGATGACAAAGGGAAGAATGTAAGCTTACAT |
| (SEQ ID NO: 40) | CAGGATTTTTTGGAACTCGAGAA |
| RNA complex_204- | AAGGATCCCTTTAAGTGGGACGATGGAAGAGGCATAGGATGACAAAGGGAAGAAGAACGGCT |
| 5p_subtype15 | GTCAAGCCGTTCAAGAAGAATAGAAGGCATAGGATGACAAAGGGAAGAACCATCGTCCCACT |
| (SEQ ID NO: 41) | TAAAGTTTTTTGGAACTCGAGAA |
| RNA complex_204- | AAGGATCCACTCTTAGTGTCCTTATGAAGAGGCATAGGATGACAAAGGGAAGAAAGATCGAA |
| 5p_subtype16 | GTCATTCGATCTAAGAAGAATAGAAGGCATAGGATGACAAAGGGAAGAACATAAGGACACTA |
| (SEQ ID NO: 42) | AGAGTTTTTTTGGAACTCGAGAA |
| RNA complex_204- | AAGGATCCGCAGCGATCGCTCGTACAAAGAGGCAUAGGATGACAAAGGGAAGAACAATCCAA |
| 5p_subtype17 | GTCATTGGATTGAAGAAGAATAGAAGGCATAGGATGACAAAGGGAAGAATGTACGAGCGATC |
| (SEQ ID NO: 43) | GCTGCTTTTTTGGAACTCGAGAA |
| RNA complex_204- | AAGGATCCTACTAAGACAGTATCTCCAAGAGGCATAGGATGACAAAGGGAAGAAGTCTCTAG |
| 5p_subtype18 | GTCACTAGAGACAAGAAGAATAGAAGGCATAGGATGACAAAGGGAAGAAGGAGATACTGTCT |
| (SEQ ID NO: 44) | TAGTATTTTTTGGAACTCGAGAA |
| RNA complex_204- | AAGGATCCTGCGGCGCGACTAACCGAAAGAGGCATAGGATGACAAAGGGAAGAAGGTCACTC |
| 5p_subtype19 | GTCAGAGTGACCAAGAAGAATAGAAGGCATAGGATGACAAAGGGAAGAATCGGTTAGTCGCG |
| (SEQ ID NO: 45) | CCGCATTTTTTGGAACTCGAGAA |
| RNA complex_204- | AAGGATCCTACCTATATCTCCACGTTAAGAGGCATAGGATGACAAAGGGAAGAAGACGAATC |
| 5p_subtype20 | GTCAGATTCGTCAAGAAGAATAGAAGGCATAGGATGACAAAGGGAAGAAAACGTGGAGATAT |
| (SEQ ID NO: 46) | AGGTATTTTTTGGAACTCGAGAA |
| RNA complex_204- | AAGGATCCGACGAGTCGTAGCGCAGAAAGAGGCATAGGATGACAAAGGGAAGAACAGTTGCT |
| 5p_subtype21 | GTCAAGCAACTGAAGAAGAATAGAAGGCATAGGATGACAAAGGGAAGAATCTGCGCTACGAC |
| (SEQ ID NO: 47) | TCGTCTTTTTTGGAACTCGAGAA |
| RNA complex_204- | AAGGATCCGTAAATCACCCTACTGCCAAGAGGCATAGGATGACAAAGGGAAGAAACTCTTAT |
| 5p_subtype22 | GTCAATAAGAGTAAGAAGAATAGAAGGCATAGGATGACAAAGGGAAGAAGGCAGTAGGGTGA |
| (SEQ ID NO: 48) | TTTACTTTTTTGGAACTCGAGAA |
| RNA complex_204- | AAGGATCCCTAAGTTGTTAATAATTGAAGAGGCATAGGATGACAAAGGGAAGAAATGTGCCG |
| 5p_subtype23 | GTCACGGCACATAAGAAGAATAGAAGGCATAGGATGACAAAGGGAAGAACAATTATTAACAA |
| (SEQ ID NO: 49) | CTTAGTTTTTTGGAACTCGAGAA |
| RNA complex_204- | AAGGATCCCAGACCAGCCAGATCGGCAAGAGGCATAGGATGACAAAGGGAAGAACGCGAACG |
| 5p_subtype24 | GTCACGTTCGCGAAGAAGAATAGAAGGCATAGGATGACAAAGGGAAGAAGCCGATCTGGCTG |
| (SEQ ID NO: 50) | GTCTGTTTTTTGGAACTCGAGAA |
| RNA complex_204- | AAGGATCCTGCGTAACGAAGTGGGGGAAGAGGCATAGGATGACAAAGGGAAGAATAGAGGAG |
| 5p_subtype25 | GTCACTCCTCTAAAGAAGAATAGAAGGCATAGGATGACAAAGGGAAGAACCCCCACTTCGTT |
| (SEQ ID NO: 51) | ACGCATTTTTTGGAACTCGAGAA |
| RNA complex_204- | AAGGATCCCACGGTCCTAGTCTTTTGAAGAGGCATAGGATGACAAAGGGAAGAATCGTCTAA |
| 5p_subtype26 | GTCATTAGACGAAAGAAGAATAGAAGGCATAGGATGACAAAGGGAAGAACAAAAGACTAGGA |
| (SEQ ID NO: 52) | CCGTGTTTTTTGGAACTCGAGAA |
| RNA complex_204- | AAGGATCCATAGACTGCGGAGGCGGTAAGAGGCATAGGATGACAAAGGGAAGAATACAAACT |
| 5p_subtype27 | GTCAAGTTTGTAAAGAAGAATAGAAGGCATAGGATGACAAAGGGAAGAAACCGCCTCCGCAG |
| (SEQ ID NO: 53) | TCTATTTTTTTGGAACTCGAGAA |
| RNA complex_204- | AAGGATCCTCAGGTTTAAACCAACCTAAGAGGCATAGGATGACAAAGGGAAGAAACTGACCT |
| 5p_subtype28 | GTCAAGGTCAGTAAGAAGAATAGAAGGCATAGGATGACAAAGGGAAGAAAGGTTGGTTTAAA |
| (SEQ ID NO: 54) | CCTGATTTTTTGGAACTCGAGAA |
| RNA complex_204- | AAGGATCCTCTGACATACTTGGGGAAAAGAGGCATAGGATGACAAAGGGAAGAAACTTACGA |
| 5p_subtype29 | GTCATCGTAAGTAAGAAGAATAGAAGGCATAGGATGACAAAGGGAAGAATTCCCCAAGTATG |
| (SEQ ID NO: 55) | TCAGATTTTTTGGAACTCGAGAA |
| RNA complex_204- | AAGGATCCAAGAGTTAAGTAGCCTGAAAGAGGCATAGGATGACAAAGGGAAGAAATCAATAG |
| 5p_subtype30 | GTCACTATTGATAAGAAGAATAGAAGGCATAGGATGACAAAGGGAAGAATCAGGCTACTTAA |
| (SEQ ID NO: 56) | CTCTTTTTTTTGGAACTCGAGAA |
| RNA complex_204- | aaggatccgacggcgctaggatcatcaacaggcataggatgacaaagggaacaagtattctg |
| 5p_subtype31 | gtcacagaatacaacaggcataggatgacaaagggaacaagatgatcctagcgccgtctttt |
| (SEQ ID NO: 57) | ttggaactcgagaa |

In some aspects, the TD construct of the present disclosure comprises a nucleotide sequence at least about 80%, at least about 85%, at least about 90%, at least about 95%, at least about 96%, at least about 97%, at least about 98%, at least about 99%, or about 100% identical to the nucleotide sequence as set forth in SEQ ID NO: 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 wherein the TD construct is capable of inhibiting miR-204-5p. In some aspects, the TD construct comprises the nucleotide sequence as set forth in SEQ ID NO: 27. In some aspects, the TD construct of the present disclosure comprises the nucleotide sequence as set forth in SEQ ID NO: 28. In some aspects, the TD construct of the present disclosure comprises the nucleotide sequence as set forth in SEQ ID NO: 29. In some aspects, the TD construct of the present disclosure comprises the nucleotide sequence as set forth in SEQ ID NO: 30. In some aspects, the TD construct of the present disclosure comprises the nucleotide sequence as set forth in SEQ ID NO: 31. In some aspects, the TD construct of the present disclosure comprises the nucleotide sequence as set forth in SEQ ID NO: 32. In some aspects, the TD construct of the present disclosure comprises the nucleotide sequence as set forth in SEQ ID NO: 33. In some aspects, the TD construct of the present disclosure comprises the nucleotide sequence as set forth in SEQ ID NO: 34. In some aspects, the TD construct of the present disclosure comprises the nucleotide sequence as set forth in SEQ ID NO: 35. In some aspects, the TD construct of the present disclosure comprises the nucleotide sequence as set forth in SEQ ID NO: 36. In some aspects, the TD construct of the present disclosure comprises the nucleotide sequence as set forth in SEQ ID NO: 37. In some aspects, the TD construct of the present disclosure comprises the nucleotide sequence as set forth in SEQ ID NO: 38. In some aspects, the TD construct of the present disclosure comprises the nucleotide sequence as set forth in SEQ ID NO: 39. In some aspects, the TD construct of the present disclosure comprises the nucleotide sequence as set forth in SEQ ID NO: 40. In some aspects, the TD construct of the present disclosure comprises the nucleotide sequence as set forth in SEQ ID NO: 41. In some aspects, the TD construct of the present disclosure comprises the nucleotide sequence as set forth in SEQ ID NO: 42. In some aspects, the TD construct of the present disclosure comprises the nucleotide sequence as set forth in SEQ ID NO: 43. In some aspects, the TD construct of the present disclosure comprises the nucleotide sequence as set forth in SEQ ID NO: 44. In some aspects, the TD construct of the present disclosure comprises the nucleotide sequence as set forth in SEQ ID NO: 45. In some aspects, the TD construct of the present disclosure comprises the nucleotide sequence as set forth in SEQ ID NO: 46. In some aspects, the TD construct of the present disclosure comprises the nucleotide sequence as set forth in SEQ ID NO: 47. In some aspects, the TD construct of the present disclosure comprises the nucleotide sequence as set forth in SEQ ID NO: 48. In some aspects, the TD construct of the present disclosure comprises the nucleotide sequence as set forth in SEQ ID NO: 49. In some aspects, the TD construct of the present disclosure comprises the nucleotide sequence as set forth in SEQ ID NO: 50. In some aspects, the TD construct of the present disclosure comprises the nucleotide sequence as set forth in SEQ ID NO: 51. In some aspects, the TD construct of the present disclosure comprises the nucleotide sequence as set forth in SEQ ID NO: 52. In some aspects, the TD construct of the present disclosure comprises the nucleotide sequence as set forth in SEQ ID NO: 53. In some aspects, the TD construct of the present disclosure comprises the nucleotide sequence as set forth in SEQ ID NO: 54. In some aspects, the TD construct of the present disclosure comprises the nucleotide sequence as set forth in SEQ ID NO: 55. In some aspects, the TD construct of the present disclosure comprises the nucleotide sequence as set forth in SEQ ID NO: 56. In some aspects, the TD construct of the present disclosure comprises the nucleotide sequence as set forth in SEQ ID NO: 57.

### IV.a Chemically Modified Polynucleotides

In some aspect, a polynucleotide of the present disclosure (e.g., a TD or a portion thereof, e.g., an MBS) comprises at least one chemically modified nucleoside and/or nucleotide. When the polynucleotides of the present invention are chemically modified the polynucleotides can be referred to as “modified polynucleotides.”

A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”).

A “nucleotide” refers to a nucleoside including a phosphate group. Modified nucleotides can be synthesized by any useful method, such as, for example, chemically, enzymatically, or recombinantly, to include one or more modified or non-natural nucleosides.

Polynucleotides can comprise a region or regions of linked nucleosides. Such regions can have variable backbone linkages. The linkages can be standard phosphodiester linkages, in which case the polynucleotides would comprise regions of nucleotides.

The modified polynucleotides disclosed herein can comprise various distinct modifications. In some embodiments, the modified polynucleotides contain one, two, or more (optionally different) nucleoside or nucleotide modifications. In some aspects, a modified polynucleotide can exhibit one or more desirable properties, e.g., improved thermal or chemical stability, reduced immunogenicity, reduced degradation, increased binding to the target microRNA, reduced non-specific binding to other microRNA or other molecules, as compared to an unmodified polynucleotide.

In some aspects, a polynucleotide of the present disclosure (e.g., a TD or a portion thereof, e.g., an MBS) is chemically modified. As used herein in reference to a polynucleotide, the terms “chemical modification” or, as appropriate, “chemically modified” refer to modification with respect to adenosine (A), guanosine (G), uridine (U), thymidine (T) or cytidine (C) ribo- or deoxyribonucleosides in one or more of their position, pattern, percent or population, including, but not limited to, its nucleobase, sugar, backbone, or any combination thereof.

In some aspects, a polynucleotide of the present disclosure (e.g., a TD or a portion thereof, e.g., an MBS) can have a uniform chemical modification of all or any of the same nucleoside type or a population of modifications produced by downward titration of the same starting modification in all or any of the same nucleoside type, or a measured percent of a chemical modification of all any of the same nucleoside type but with random incorporation In another aspect, the polynucleotide of the present disclosure (e.g., a TD) can have a uniform chemical modification of two, three, or four of the same nucleoside type throughout the entire polynucleotide (such as all uridines and/or all cytidines, etc. are modified in the same way).

Modified nucleotide base pairing encompasses not only the standard adenine-thymine, adenine-uracil, or guanine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleobase inosine and adenine, cytosine or uracil. Any combination of base/sugar or linker can be incorporated into polynucleotides of the present disclosure.

The skilled artisan will appreciate that, except where otherwise noted, polynucleotide sequences set forth in the instant application will recite “T”s in a representative DNA sequence but where the sequence represents RNA, the “T”s would be substituted for “U”s. For example, TD's of the present disclosure can be administered as RNAs, as DNAs, or as hybrid molecules comprising both RNA and DNA units.

In some aspects, the polynucleotide (e.g., a TD or a portion thereof, e.g., an MBS) includes a combination of at least two (e.g., 2, 3, 4, 5, 6, 7, 8, 8, 10, 11, 12, 13, 14, 15, 16, 17, 18, 18, 20 or more) modified nucleobases.

In some aspects, the nucleobases, sugar, backbone linkages, or any combination thereof in a polynucleotide (e.g., a TD or a portion thereof, e.g., an MBS) are modified by at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100%.

### (i) Base Modifications

In certain aspects, the chemical modification is at nucleobases in a polynucleotide of the present disclosure (e.g., a TD or a portion thereof, e.g., an MBS). In some aspects, the at least one chemically modified nucleoside is a modified uridine (e.g., pseudouridine (w), 2-thiouridine (s2U), 1-methyl-pseudouridine (m1ψ), 1-ethyl-pseudouridine (e1ψ), or 5-methoxy-uridine (mo5U)), a modified cytosine (e.g., 5-methyl-cytidine (m5C)) a modified adenosine (e.g, 1-methyl-adenosine (m1A), N6-methyl-adenosine (m6A), or 2-methyl-adenine (m2A)), a modified guanosine (e.g., 7-methyl-guanosine (m7G) or 1-methyl-guanosine (m1G)), or a combination thereof.

In some aspects, the polynucleotide of the present disclosure (e.g., a TD or a portion thereof, e.g., an MBS) is uniformly modified (e.g., fully modified, modified throughout the entire sequence) for a particular modification. For example, a polynucleotide can be uniformly modified with the same type of base modification, e.g., 5-methyl-cytidine (m5C), meaning that all cytosine residues in the polynucleotide sequence are replaced with 5-methyl-cytidine (m5C). Similarly, a polynucleotide can be uniformly modified for any type of nucleoside residue present in the sequence by replacement with a modified nucleoside such as any of those set forth above.

In some aspects, the polynucleotide of the present disclosure (e.g., a TD or a portion thereof, e.g., an MBS) includes a combination of at least two (e.g., 2, 3, 4 or more) of modified nucleobases. In some aspects, at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of a type of nucleobases in a polynucleotide of the present disclosure (e.g., a TD or a portion thereof, e.g., an MBS) are modified nucleobases.

### (ii) Backbone Modifications

In some aspects, the polynucleotide of the present disclosure (e.g., a TD or a portion thereof, e.g., an MBS) can include any useful linkage between the nucleosides. Such linkages, including backbone modifications, that are useful in the composition of the present disclosure include, but are not limited to the following: 3′-alkylene phosphonates, 3′-amino phosphoramidate, alkene containing backbones, aminoalkylphosphoramidates, aminoalkylphosphotriesters, boranophosphates, —CH₂—O—N(CH₃)—CH₂—, —CH₂—N(CH₃)—N(CH₃)—CH₂—, —CH₂—NH—CH₂—, chiral phosphonates, chiral phosphorothioates, formacetyl and thioformacetyl backbones, methylene (methylimino), methylene formacetyl and thioformacetyl backbones, methyleneimino and methylenehydrazino backbones, morpholino linkages, —N(CH₃)—CH₂—CH₂—, oligonucleosides with heteroatom internucleoside linkage, phosphinates, phosphoramidates, phosphorodithioates, phosphorothioate internucleoside linkages, phosphorothioates, phosphotriesters, PNA, siloxane backbones, sulfamate backbones, sulfide sulfoxide and sulfone backbones, sulfonate and sulfonamide backbones, thionoalkylphosphonates, thionoalkylphosphotriesters, and thionophosphoramidates.

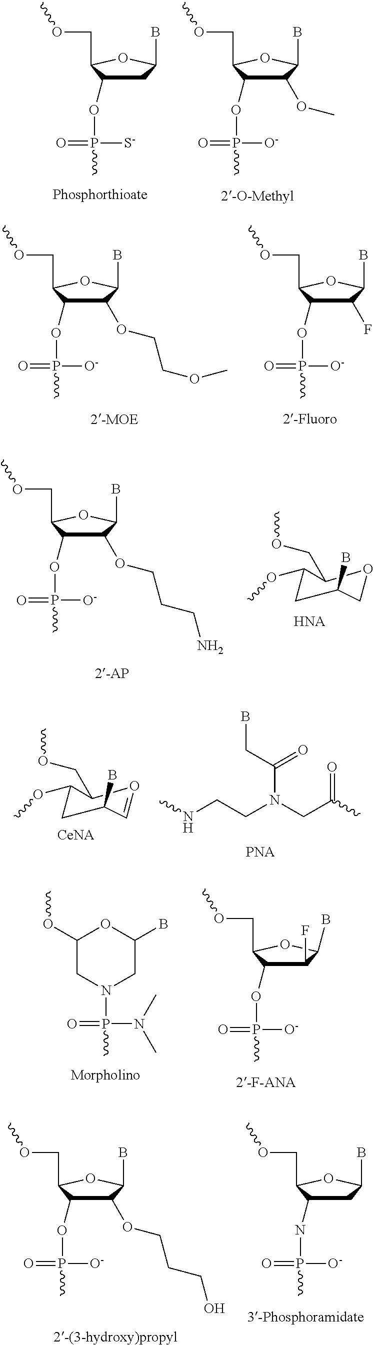

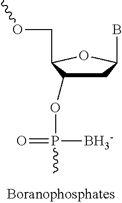

In some aspects, the presence of a backbone linkage disclosed above increase the stability and resistance to degradation of a polynucleotide of the present disclosure (e.g., a TD or a portion thereof, e.g., an MBS).

In some aspects, at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of the backbone linkages in a polynucleotide of the present disclosure (e.g., a TD or a portion thereof, e.g., an MBS) are modified (e.g., all of them are phosphorothioate).

In some aspects, 1, 2, 3, 4, 5, 6, 7, 8, 9, 10, 11, 12, 13, 14, 15, 16, 17, 18, 19, 20, or 21 backbone linkages in a polynucleotide of the present disclosure (e.g., a TD or a portion thereof, e.g., an MBS) are modified (e.g., phosphorothioate).

(iii) Sugar Modifications

The modified nucleosides and nucleotides which can be incorporated into a polynucleotide of the present disclosure (e.g., a TD or a portion thereof, e.g., an MBS), can be modified on the sugar of the nucleic acid. In some aspects, the sugar modification increases the affinity of the binding of a MBS to its target miRNA. Incorporating affinity-enhancing nucleotide analogues in the MBS, such as LNA or 2′-substituted sugars can allow the length of MBS to be reduced, and also may reduce the upper limit of the size an MBS before non-specific or aberrant binding takes place.

In some aspects, at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of the nucleotides in a polynucleotide of the present disclosure (e.g., a TD or a portion thereof, e.g., an MBS) contain sugar modifications (e.g., LNA).

In some aspects, 1, 2, 3, 4, 5, 6, 7, 8, 9, 10, 11, 12, 13, 14, 15, 16, 17, 18, 19, 20, 21, or 22 nucleotide units in a polynucleotide of the present disclosure (e.g., a TD or a portion thereof, e.g., an MBS) are sugar modified (e.g., LNA).

Generally, RNA includes the sugar group ribose, which is a 5-membered ring having an oxygen. Exemplary, non-limiting modified nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with a-L-threofuranosyl-(3′→2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar.

The 2′ hydroxyl group (OH) of ribose can be modified or replaced with a number of different substituents. Exemplary substitutions at the 2′-position include, but are not limited to, H, halo, optionally substituted C₁₋₆ alkyl; optionally substituted C₁₋₆ alkoxy; optionally substituted C₆₋₁₀ aryloxy; optionally substituted C₃₋₈ cycloalkyl; optionally substituted C₃₋₈ cycloalkoxy; optionally substituted C₆₋₁₀ aryloxy; optionally substituted C₆₋₁₀ aryl-C₁₋₆ alkoxy, optionally substituted C₁₋₁₂ (heterocyclyl)oxy; a sugar (e.g., ribose, pentose, or any described herein); a polyethyleneglycol (PEG), —O(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C₁₋₆ alkylene or C₁₋₆ heteroalkylene bridge to the 4′-carbon of the same ribose sugar, where exemplary bridges include methylene, propylene, ether, amino bridges, aminoalkyl, aminoalkoxy, amino, and amino acid.

In some aspects, nucleotide analogues present in a polynucleotide of the present disclosure (e.g., a TD or a portion thereof, e.g., an MBS) comprise, e.g., 2′-O-alkyl-RNA units, 2′-OMe-RNA units, 2′-O-alkyl-SNA, 2′-amino-DNA units, 2′-fluoro-DNA units, LNA units, arabino nucleic acid (ANA) units, 2′-fluoro-ANA units, HNA units, INA (intercalating nucleic acid) units, 2′MOE units, or any combination thereof. In some aspects, the LNA is, e.g., oxy-LNA (such as beta-D-oxy-LNA, or alpha-L-oxy-LNA), amino-LNA (such as beta-D-amino-LNA or alpha-L-amino-LNA), thio-LNA (such as beta-D-thio0-LNA or alpha-L-thio-LNA), ENA (such a beta-D-ENA or alpha-L-ENA), or any combination thereof.

In some aspects, a polynucleotide of the present disclosure (e.g., a or a portion thereof, e.g., an MBS) can comprise both modified RNA nucleotide analogues (e.g., LNA) and DNA units. In some aspects, an MBS of the present disclosure is a gapmer. See, e.g., U.S. Pat. Nos. 8,404,649; 8,580,756; 8,163,708; 9,034,837; all of which are herein incorporated by reference in their entireties. In some aspects, an MBS of the present disclosure is a micromir. See U.S. Pat. Appl. Publ. No. US20180201928, which is herein incorporated by reference in its entirety.

### V. Pharmaceutical Compositions

The present disclosure also provides pharmaceutical compositions comprising vectors, e.g., AAV vectors, or polynucleotides of the present disclosure that are suitable for administration to a subject. The pharmaceutical compositions generally comprise a vector, e.g., an AAV vector, or a polynucleotide of the present disclosure and a pharmaceutically-acceptable excipient or carrier in a form suitable for administration to a subject. Pharmaceutically acceptable excipients or carriers are determined in part by the particular composition being administered, as well as by the particular method used to administer the composition.

Accordingly, there is a wide variety of suitable formulations of pharmaceutical compositions comprising a plurality of EVs (e.g., exosomes). (See, e.g., Remington's Pharmaceutical Sciences, Mack Publishing Co., Easton, Pa. 18th ed. (1990)). The pharmaceutical compositions are generally formulated sterile and in full compliance with all Good Manufacturing Practice (GMP) regulations of the U.S. Food and Drug Administration. In some aspects, the pharmaceutical composition comprises one or more vectors, e.g., AAV vector, or polynucleotides described herein.

In some aspects, a pharmaceutical composition comprises one or more therapeutic agents and one or more vectors, e.g., AAV vector, or polynucleotides described herein. In certain aspects, the vectors, e.g., AAV vector, and polynucleotides described herein are co-administered with of one or more additional therapeutic agents, in a pharmaceutically acceptable carrier. In some aspects, the pharmaceutical composition comprising the vectors, e.g., AAV vector, or polynucleotides described herein is administered prior to administration of the additional therapeutic agent(s). In other aspects, the pharmaceutical composition comprising the vectors, e.g., AAV vector, or polynucleotides described herein is administered after the administration of the additional therapeutic agent(s). In further aspects, the pharmaceutical composition comprising the vectors, e.g., AAV vector, or polynucleotides described herein is administered concurrently with the additional therapeutic agent(s).

Provided herein are pharmaceutical compositions comprising vectors, e.g., AAV vector, or polynucleotides described herein having the desired degree of purity, and a pharmaceutically acceptable carrier or excipient, in a form suitable for administration to a subject. Pharmaceutically acceptable excipients or carriers can be determined in part by the particular composition being administered, as well as by the particular method used to administer the composition. Accordingly, there is a wide variety of suitable formulations of pharmaceutical compositions comprising a plurality of vectors, e.g., AAV vectors, or polynucleotides described herein. (See, e.g., Remington's Pharmaceutical Sciences, Mack Publishing Co., Easton, Pa. 21st ed. (2005)). The pharmaceutical compositions are generally formulated sterile and in full compliance with all Good Manufacturing Practice (GMP) regulations of the U.S. Food and Drug Administration.

In some aspects, a pharmaceutical composition comprises one or more therapeutic agents and a vector, e.g., AAV vector, or polynucleotide described herein. In certain aspects, the vectors, e.g., AAV vector, or polynucleotides described herein are co-administered with of one or more additional therapeutic agents, in a pharmaceutically acceptable carrier. In some aspects, the pharmaceutical composition comprising vectors, e.g., AAV vector, or polynucleotides described herein is administered prior to administration of the additional therapeutic agents.

In other aspects, the pharmaceutical composition comprising vectors, e.g., AAV vector, or polynucleotides described herein is administered after the administration of the additional therapeutic agents. In further aspects, the pharmaceutical composition comprising vectors, e.g., AAV vector, or polynucleotides described herein is administered concurrently with the additional therapeutic agents.

Acceptable carriers, excipients, or stabilizers are nontoxic to recipients (e.g., animals or humans) at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Examples of carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the vectors, e.g., AAV vector, or polynucleotides described herein, use thereof in the compositions is contemplated. Supplementary therapeutic agents can also be incorporated into the compositions. Typically, a pharmaceutical composition is formulated to be compatible with its intended route of administration. The vectors, e.g., AAV vector, or polynucleotides described herein can be administered by parenteral, topical, intravenous, oral, subcutaneous, intra-arterial, intradermal, transdermal, rectal, intracranial, intraperitoneal, intranasal, intratumoral, intramuscular route or as inhalants. In certain aspects, the pharmaceutical composition comprising vectors, e.g., AAV vector, or polynucleotides described herein is administered intravenously, e.g. by injection. The vectors, e.g., AAV vector, or polynucleotides described herein can optionally be administered in combination with other therapeutic agents that are at least partly effective in treating the disease, disorder or condition for which the vectors, e.g., AAV vector, or polynucleotides described herein are intended.

Solutions or suspensions can include the following components: a sterile diluent such as water, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfate; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The preparation can be enclosed in ampoules, disposable syringes or multiple dose vials made of glass or plastic.

Pharmaceutical compositions suitable for injectable use include sterile aqueous solutions (if water soluble) or dispersions and sterile powders. For intravenous administration, suitable carriers include physiological saline, bacteriostatic water, Cremophor EL™ (BASF, Parsippany, N.J.) or phosphate buffered saline (PBS). The composition is generally sterile and fluid to the extent that easy syringeability exists. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. If desired, isotonic compounds, e.g., sugars, polyalcohols such as mannitol, sorbitol, and sodium chloride can be added to the composition. Prolonged absorption of the injectable compositions can be brought about by including in the composition a compound which delays absorption, e.g., aluminum monostearate and gelatin.

Sterile injectable solutions can be prepared by incorporating the vectors, e.g., AAV vector, or polynucleotides described herein in an effective amount and in an appropriate solvent with one or a combination of ingredients enumerated herein, as desired. Generally, dispersions are prepared by incorporating the vectors, e.g., AAV vector, or polynucleotides described herein into a sterile vehicle that contains a basic dispersion medium and any desired other ingredients. In the case of sterile powders for the preparation of sterile injectable solutions, methods of preparation are vacuum drying and freeze-drying that yields a powder of the active ingredient plus any additional desired ingredient from a previously sterile-filtered solution thereof. The vectors, e.g., AAV vector, or polynucleotides described herein can be administered in the form of a depot injection or implant preparation which can be formulated in such a manner to permit a sustained or pulsatile release of the vectors, e.g., AAV vector, or polynucleotides described herein.

Systemic administration of compositions comprising vectors, e.g., AAV vector, or polynucleotides described herein can also be by transmucosal means. For transmucosal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of, e.g., nasal sprays.

In certain aspects the pharmaceutical composition comprising vectors, e.g., AAV vector, or polynucleotides described herein of the present disclosure is administered intravenously into a subject that would benefit from the pharmaceutical composition. In certain other aspects, the composition is administered to the lymphatic system, e.g., by intralymphatic injection or by intranodal injection (see e.g., Senti et al., PNAS 105(46): 17908 (2008)), or by intramuscular injection, by subcutaneous administration, by intratumoral injection, by direct injection into the thymus, or into the liver.

In certain aspects, the pharmaceutical composition comprising vectors, e.g., AAV vector, or polynucleotides described herein is administered as a liquid suspension. In certain aspects, the pharmaceutical composition is administered as a formulation that is capable of forming a depot following administration. In certain preferred aspects, the depot slowly releases the vectors, e.g., AAV vector, or polynucleotides described herein into circulation, or remains in depot form.

Typically, pharmaceutically-acceptable compositions are highly purified to be free of contaminants, are biocompatible and not toxic, and are suited to administration to a subject. If water is a constituent of the carrier, the water is highly purified and processed to be free of contaminants, e.g., endotoxins.

The pharmaceutically-acceptable carrier can be lactose, dextrose, sucrose, sorbitol, mannitol, starch, gum acacia, calcium phosphate, alginates, gelatin, calcium silicate, micro-crystalline cellulose, polyvinylpyrrolidone, cellulose, water, syrup, methyl cellulose, methylhydroxy benzoate, propylhydroxy benzoate, talc, magnesium stearate, and/or mineral oil, but is not limited thereto. The pharmaceutical composition can further include a lubricant, a wetting agent, a sweetener, a flavor enhancer, an emulsifying agent, a suspension agent, and/or a preservative.

The pharmaceutical compositions described herein comprise the vectors, e.g., AAV vector, or polynucleotides described herein and optionally a pharmaceutically active or therapeutic agent. The therapeutic agent can be a biological agent, a small molecule agent, or a nucleic acid agent.

Dosage forms are provided that comprise vectors, e.g., AAV vectors, polynucleotides, or pharmaceutical compositions described herein. In some aspects, the dosage form is formulated as a liquid suspension for intravenous injection.

The vector, e.g., an AAV vector, polynucleotide, or pharmaceutical composition may be used concurrently with other drugs. To be specific, the vectors, e.g., an AAV vector, polynucleotides, or pharmaceutical compositions of the present disclosure may be used together with medicaments such as hormonal therapeutic agents, chemotherapeutic agents, immunotherapeutic agents, medicaments inhibiting the action of cell growth factors or cell growth factor receptors and the like.

### VI. Kits

The present disclosure also provides kits, or products of manufacture, comprising a vector, e.g., an AAV vector, a polynucleotide, or a pharmaceutical composition of the present disclosure and optionally instructions for use. In some aspects, the kit or product of manufacture comprises a vector, e.g., an AAV vector, a polynucleotide, or a pharmaceutical composition of the present disclosure in one or more containers. In some aspects, the kit or product of manufacture comprises a vector, e.g., an AAV vector, a polynucleotide, or a pharmaceutical composition of the present disclosure and a brochure. In some aspects, the kit or product of manufacture comprises a vector, e.g., an AAV vector, a polynucleotide, or a pharmaceutical composition of the present disclosure and instructions for use. One skilled in the art will readily recognize that vectors, polynucleotides, and pharmaceutical compositions of the present disclosure, or combinations thereof, can be readily incorporated into one of the established kit formats which are well known in the art.

### Sequences

| SEQ | | |
|---|---|---|
| ID NO | Description | Sequence |
| 1 | miR204-5p(RNA) | 5′uucccuuugucauccuaugccu3′ |
| | Seed region underlined | |
| 2 | miR204-5p(RNA) complement | 5′aggcauaggaugacaaagggaa3′ |
| | miR204-5p binding site | |
| | Sequence complementary to | |
| | seed region underlined | |
| 3 | miR204-5p (DNA) | 5′ttccctttgtcatcctatgcct3′ |
| | DNA encoding miR204-5p | |
| | binding site | |
| 4 | miR204-5p (DNA) complement | 5′aggcataggatgacaaagggaa3′ |
| 5 | miR204-3p (RNA) | 5′gcugggaaggcaaagggacgu3′ |
| | Seed region underlined | |
| 6 | miR204-3p (RNA) complement | 5′acgucccuuugccuucccagc3′ |
| | miR204-3p binding site | |
| | Sequence complementary to | |
| | seed region underlined | |
| 7 | miR204-3p (DNA) | 5′gctgggaaggcaaagggacgt3′ |
| | DNA encoding miR204-3p | |
| | binding site | |
| 8 | miR204-3p (DNA) complement | 5′acgtccctttgccttcccagc3′ |
| 9 | Stem 2 (RNA) | 5′guauucug3′ |
| 10 | Stem 2 (DNA) | 5′gtattctg3′ |
| 11 | Stem 2 complement (RNA) | 5′cagaauac3′ |
| 12 | Stem 2 complement (DNA) | 5′cagaatac3′ |
| 13 | Loop (RNA) | 5′guca3′ |
| 14 | Loop (DNA) | 5′gtca3′ |
| 15 | Stem 1 (RNA) | 5′gacggcgcuaggaucauc3′ |
| 16 | Stem 1 (DNA) | 5′gacggcgctaggatcatc3′ |
| 17 | Stem 1 complement (RNA) | 5′gaugauccuagctccguc3′ |
| 18 | Stem 1 complement (DNA) | 5′gatgatcctagctccgtc3′ |
| 19 | 5′ Spacer (DNA/RNA) | 5′aac3′ |
| 20 | 3′ Spacer (DNA/RNA) | 5′caa3′ |
| 21 | Linker (RNA) | 5′aacaauac3′ |
| 22 | Linker (DNA) | 5′-aacaatac-3′ |
| 23 | miR204-5p TD construct | 5′- |
| | miRNA binding sites double | aaggatccgacggcgctaggatcatcaacaggcataggat |
| | underlined | gacaaagggaacaagtattctggtcacagaatacaacagg |
| | | cataggatgacaaagggaacaagatgatcctagcgccgtc |
| | | ttttttggaaaagcttaa-3′ |
| 24 | miR416 Control construct | 5′- |
| | miRNA binding sites double | aaggatccgacggcgctaggatcatcaacggttcgtacgt |
| | underlined | acactgttcacaagtattctggtcacagaatacaacggtt |
| | | cgtacgtacactgttcacaagatgatcctagcgccgtctt |
| | | ttttggaaaagcttaa-3′ |
| 25 | miR204-5p seed sequence | 5′uucccuu3′ |
| 26 | miR204-3p seed sequence | 5′gcuggga3′ |

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986);); Crooke, Antisense drug Technology: Principles, Strategies and Applications, 2nd Ed. CRC Press (2007) and in Ausubel et al. (1989) Current Protocols in Molecular Biology (John Wiley and Sons, Baltimore, Md.).

All of the references cited above, as well as all references cited herein, are incorporated herein by reference in their entireties.

The following examples are offered by way of illustration and not by way of limitation.

### EXAMPLES

### Example 1

### Data Analysis

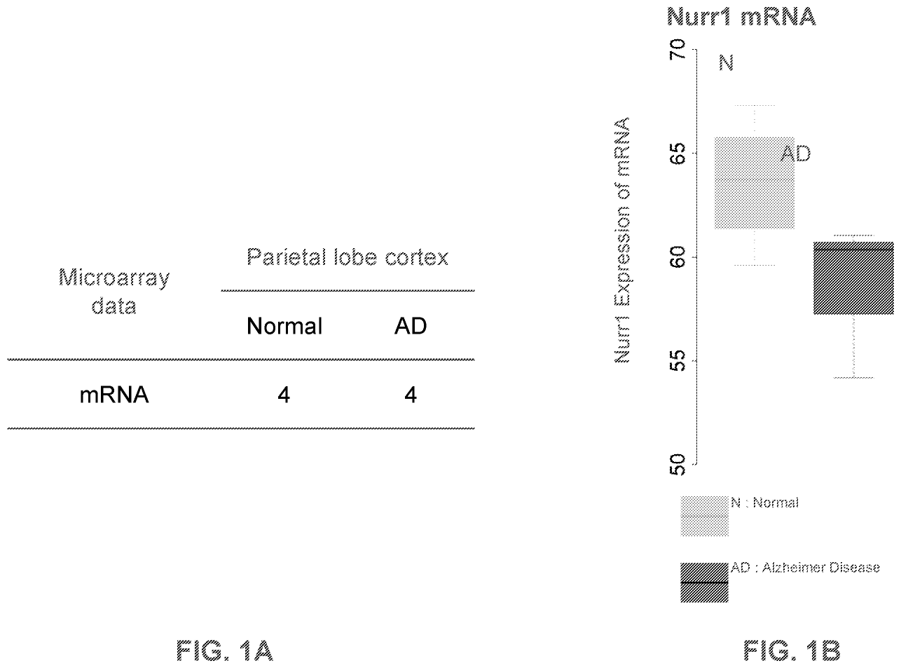

The expression data generated by this study are available in the NCBI Gene Expression Omnibus (GEO) as accession GSE16759. FIGS. 1A and 1B show an analysis of mRNA microarray data from 4 age-matched controls and 4 AD patients in accession number GES16759. FIG. 1A shows sample information. FIG. 1B shows that AD patient's tissues show lower level of Nurr1 mRNA compared to those of normal tissues

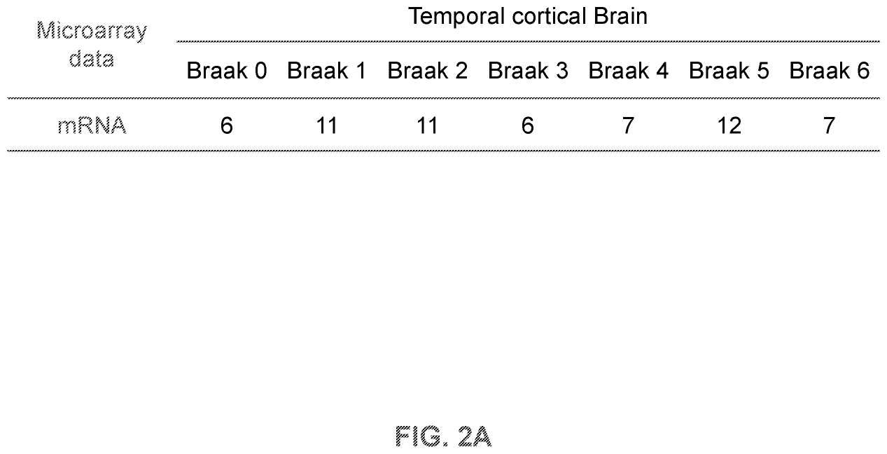

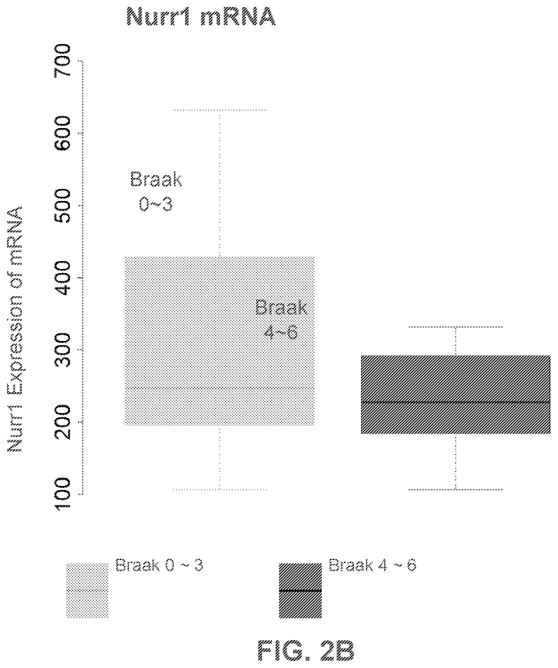

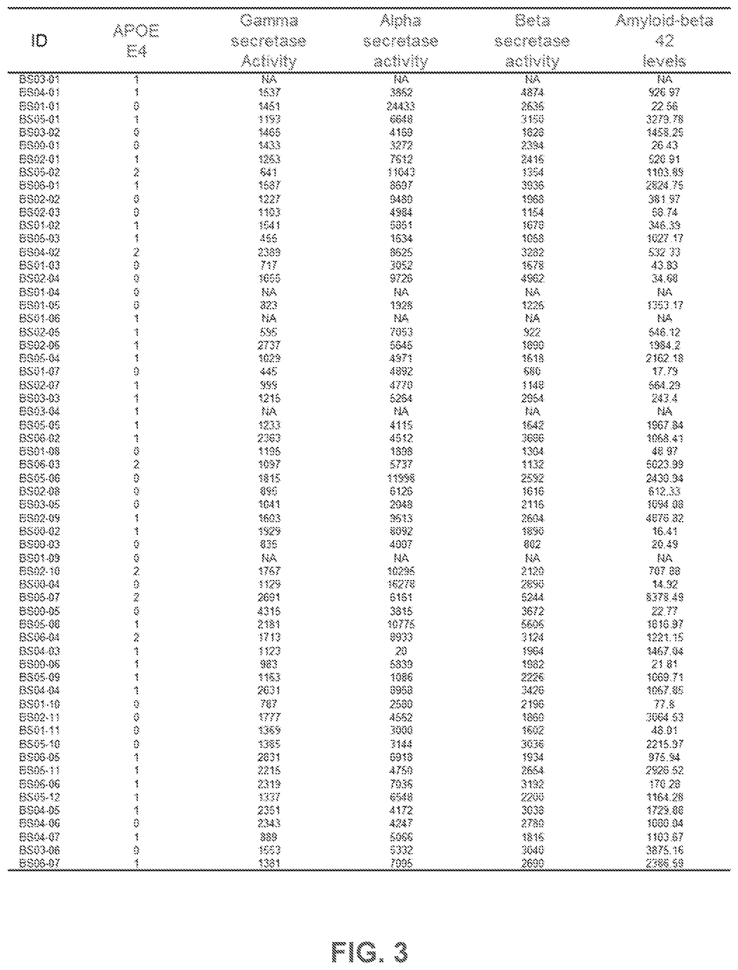

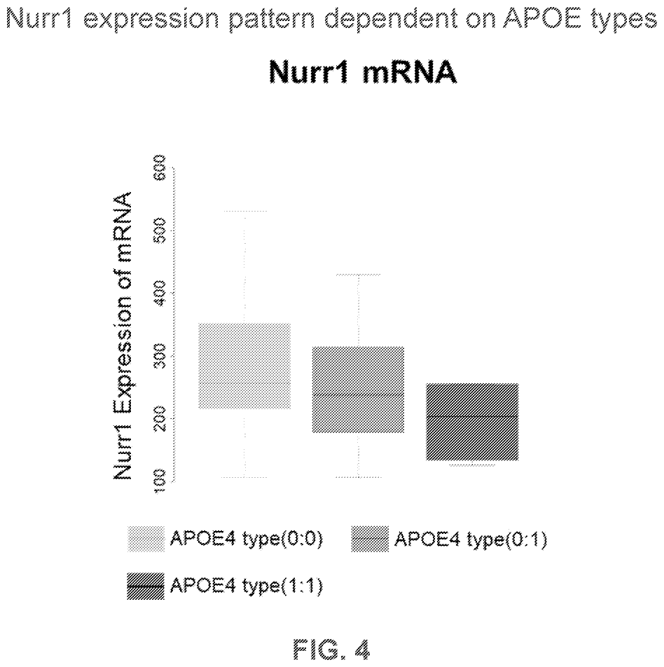

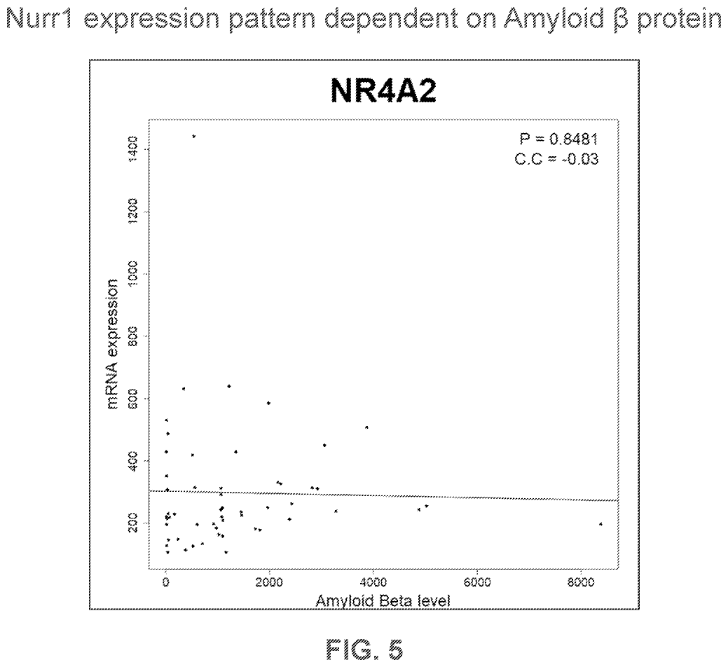

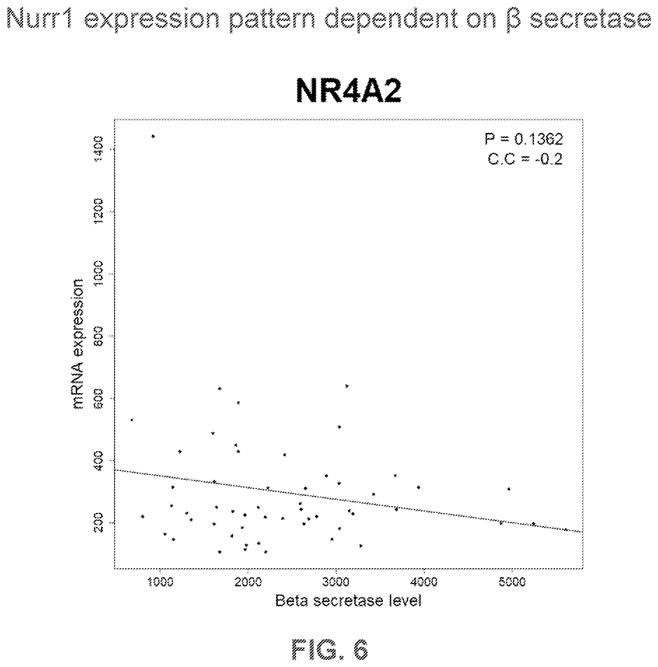

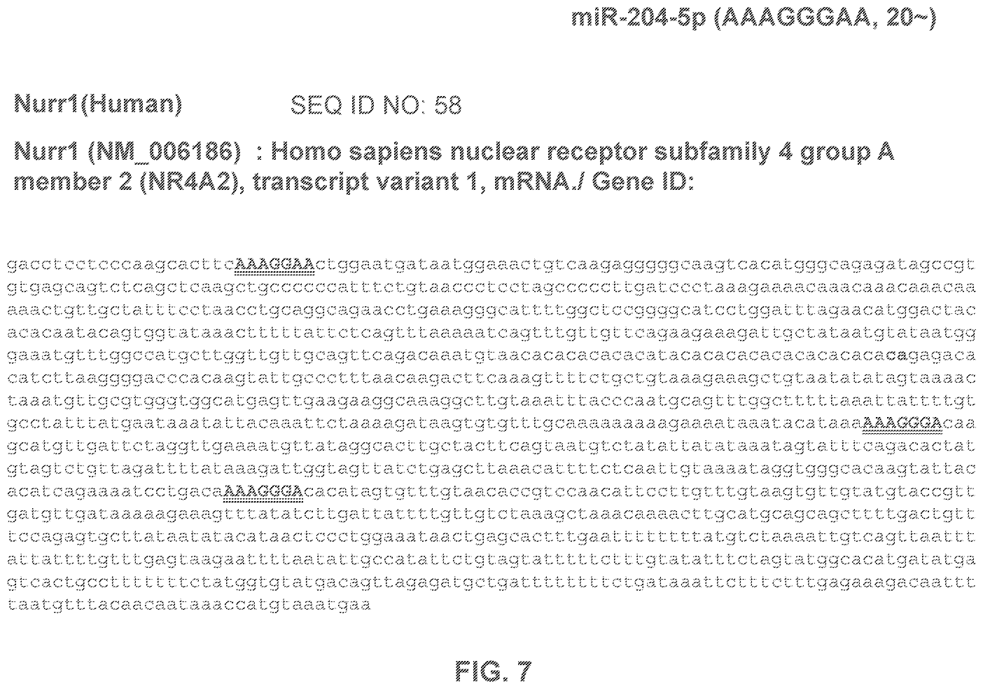

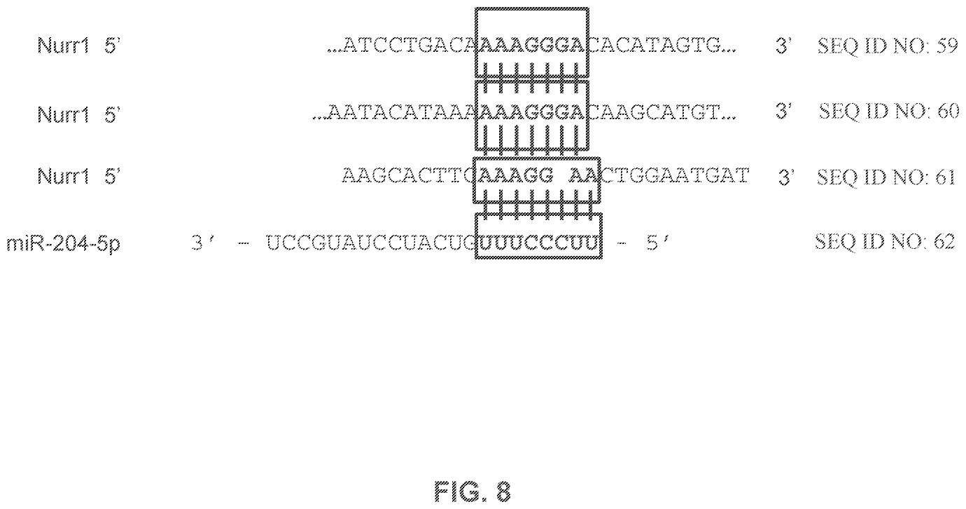

The microarray data have been deposited in NCBI's Gene Expression Omnibus (Edgar, 2002) and are accessible through GEO Series accession number GSE106241. The mass spectrometry proteomics data have been deposited to the ProteomeXchange Consortium via the PRIDE partner repository with the dataset identifier PXD008016 (Vizcaino et al., 2016). FIGS. 1A, 1B, 2A, 2B, 3, 4 and 5 are derived from the analysis of the above data.

### Example 2

### Luciferase Reporter Assays

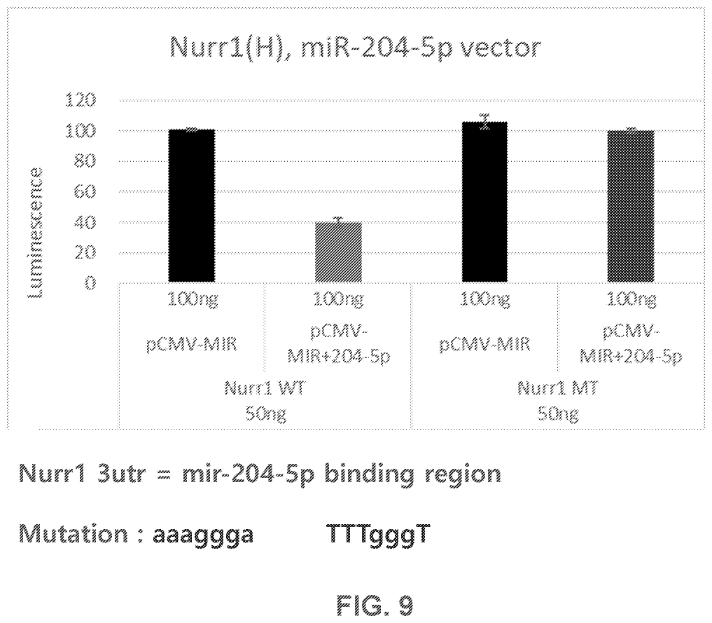

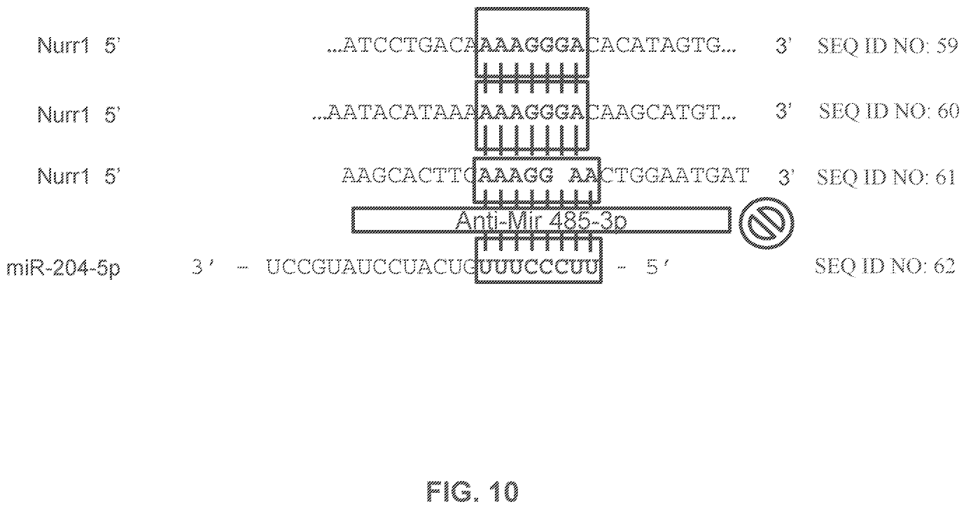

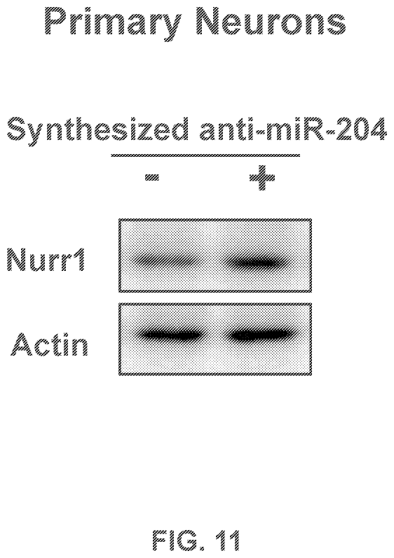

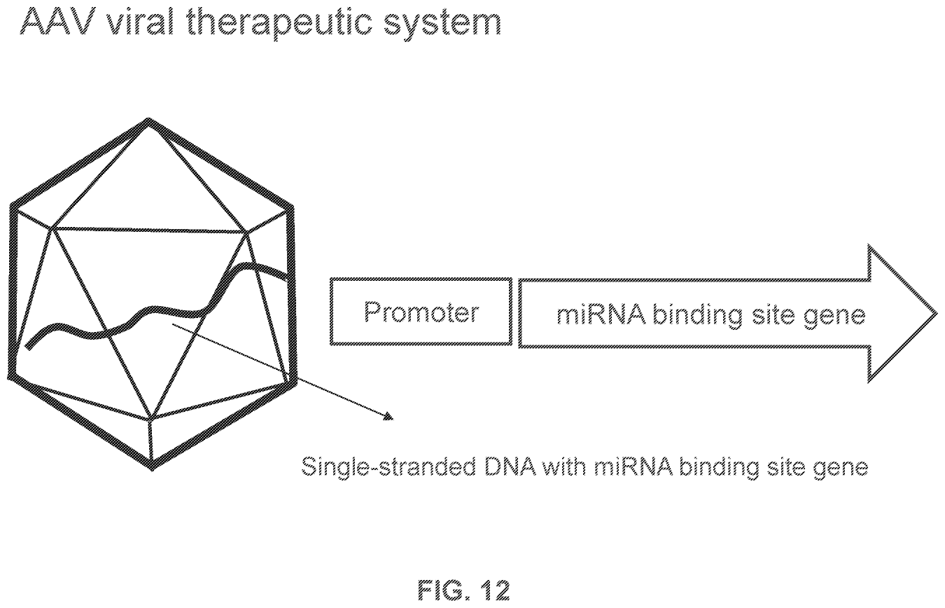

Sequence of segments with wide-type (WT) 3′-UTR region of Nurr1 mRNA containing the predicted miR-204-5p binding sequences or mutant 3′-UTR (aaaggga was mutated to tttgggt) were PCR amplified and cloned into the psiCHECK-2 luciverase reporter vector (Promega, Madison, Wis., USA). See FIG. 9. For the luciferase activity assay, HEK 293 T cells were plated into 24-well plates and co-transfected with psiCHECK2-Nurr1-3′ UTR-WT or psiCHECK2-Nurr1-3′UTR-MT, with pCMV-MIR(Origene), pCMV-MIR-miR-204-5p. After 48 h of transfection, firefly and Renilla luciferase activity was determined by the Dual-Luciferase Reporter Assay System (Promega). Relative Renilla luciferase activity was measured by normalizing to the firefly luciferase activity. FIG. 9 shows that when pCMV miR 204-5p, which expresses a miR-204 binding site, was added to the vector containing the wild type 3′ UTR of the Nurr1, it reduced the luminescence by about 60%. However, when the pCMV-miR 204-5p was added to a vector containing a mutant 3′ UTR of Nurr1, it did not reduce the luminescence activity compared to the negative control.

### Example 3

### Generation of Constructs

Virus constructs: For construction of Tough Decoy (TD) miR-204-5p plasmid, DNA sequence

(SEQ ID NO: 64)
(5-aactcgaggttcgatacaggggcatcaagaggcataggatgacaaag
ggaagaatttggaacgtcagttccaaaaagaagaatagaaggcataggat
gacaaagggaagaagatgcccctgtatcgaacttattggaactcgagaa
-3)

containing stem, stem loop, and two miR-204-5p binding sites by Xho1/Xho1 sites were synthesized and cloned into Xho1/Xho1 sites of pAAV-IRES-GFP vector (a purchased from CELL BIOLABS, Inc., San Diego, USA) plasmid, where CMV promoter drives expression of small RNA efficiently. DNA sequence

(SEQ ID NO: 65)
(5-aactcgaggttcgatacaggggcatcaagaagaggcttgcacagtgc
attgaatttggaacgtcagttccaaaaagaagaatagaaagaggcttgca
cagtgcattgaagatgcccctgtatcgaacttftttggaactcgagaa-
3)

containing stem, stem loop, and two scramble sequence binding sites flanked by Xho1/Xho1 sites were generated for TD control plasmid construction to serve as a non-specific control. Viral titers were 1×10⁹ IFU/ml for AAV TD control and 2×10⁹ IFU/ml for AAV TD miR-204-5p.

Subjects: SXFAD APP transgenic mice (Stock number:000664) were purchased from the Jackson Laboratory. TG and age-matched wild type (WT) littermates were used in the studies. All of the animals were kept in individually cages in a 12/12-h light/dark cycle with controlled temperature and humidity and food and water.

Stereotactic injection: All animals were initially anesthetized with 3-5% isoflurane in oxygen and fixed on stereotaxic frame (JeongDo). The AAV2 was stereotactically injected with 2.5 ul (titer of 1×109 TU/ml) into the ICV (AP: −2 mm, ML: ±1.2 mm, DV: −1.5 mm from bregma). Primary Cortical Neuron culture, transfection and Western blot

Primary cortical cultures were prepared from embryonic day (E) 18-19 mouse cortex. Neurons were transfected at 9 d in vitro (DIV) using a TransIT-X2® Transfection Reagent (Minis Bio). Neuron lysed in ice-cold RIPA buffer containing protease inhibitors and were centrifuged at 12,000 r.p.m. for 30 min at 4° C., and supernatants were collected. The samples were separated by SDS-polyacrylamide gel electrophoresis, transferred to PVDF membranes and incubated with the following primary antibodies: mouse anti-Nurr1 (Santa Cruz, Cat #sc-376984) and anti-actin (Santa Cruz, Cat #sc-47778). After behavioral test, hippocampal regions and Cortex regions were dissected from H/I mice, and brain tissue homogenized in ice-cold RIPA buffer containing protease inhibitors. Homogenates were centrifuged at 12,000 r.p.m. for 30 min at 4° C., and supernatants were collected. The results were visualized using an enhanced chemiluminescence system, and quantified by densitometric analysis (Image J software, NIH). All experiments were performed independently at least three times.

Immunohistochemistry: For immunohistochemistry, brains were removed, postfixed and embedded in paraffin. Coronal sections (10-μm thick) through the infarct were cut using a microtome and mounted on slides. The paraffin was removed, and the sections were washed with PBS-T and blocked in 10% bovine serum albumin for 2 h. Thereafter, the following primary antibodies were applied: mouse anti-Nurr1 (Santa Cruz, Cat #sc-376984), Rabbit anti-NeuN (Abcam, Cat #EPR12763) and anti-amyloid beta (BioLegend, clone 6E10). After behavioral test, hippocampal regions and Cortex regions were dissected from H/I mice, and brain tissue homogenized in ice-cold RIPA buffer containing protease inhibitors. Homogenates were centrifuged at 12,000 r.p.m. for 30 min at 4° C., and supernatants were collected. The results were visualized using an enhanced chemiluminescence system, and quantified by densitometric analysis (Image J software, NIH). All experiments were performed independently at least three times.

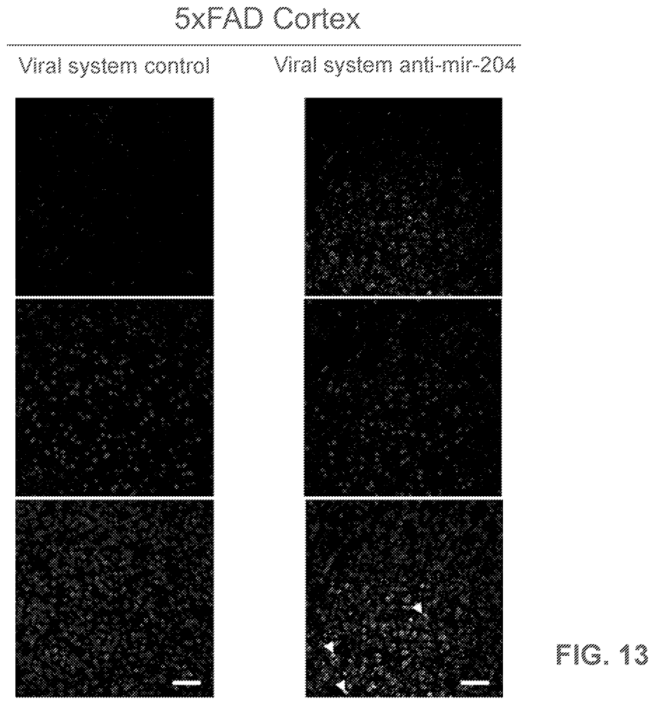

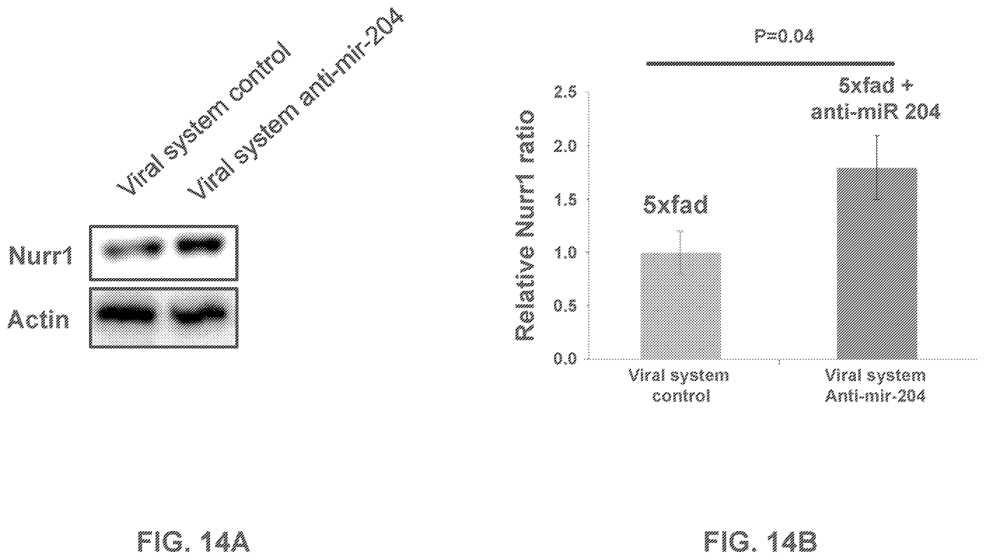

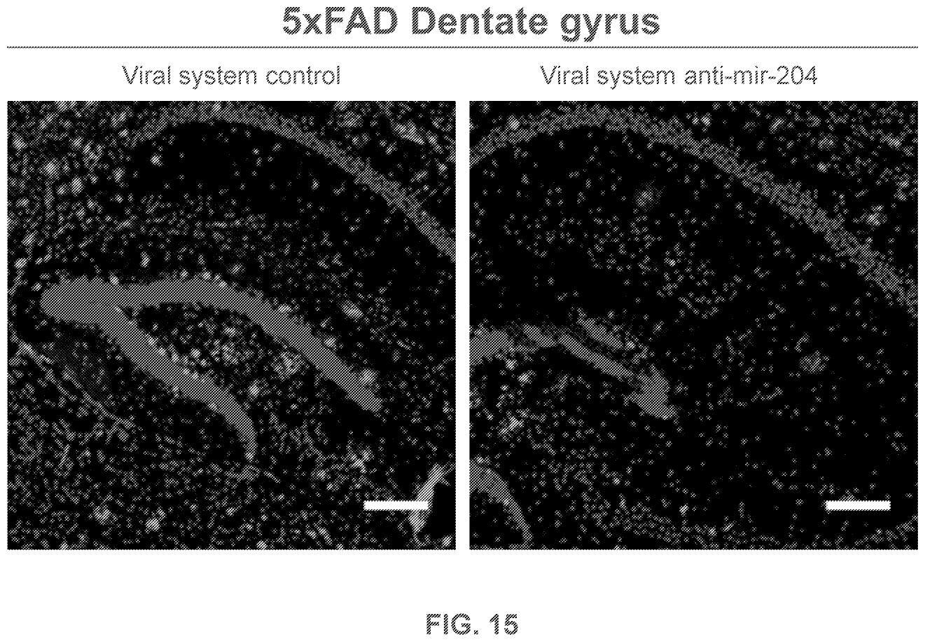

FIG. 13 shows representative cortical images from confocal imaging of Nurr1. Viral system anti-miR-204 delivery in cortex of 5×FAD mice increases Nurr1. Representative cortical images from confocal imaging of triple staining for Nurr1 (green-top), Neuron (red-middle), and Nucleus (blue-bottom). FIGS. 14A and 14B show immunoblot detection of Nurr1 proteins in brain lysates of control Mock- or Viral system anti-miR-204-5p-treated 5×FAD. Viral system anti-miR 204-5p promotes Nurr1 expression in 5×FAD brain. FIG. 15 shows immunohistochemical analysis of dentate gyrus of 5×FAD. Viral system anti-miR-204 decreases amyloid plaque burden in 5×FAD. Immunohistochemical analysis of dentate gyrus after administration of mock or Viral system anti-miR-204. Diffuse plaques in the brain sections were stained by anti-amyloid beta (clone 6E10, red color) and nucleus (blue).

### Example 4

### Behavior Tests

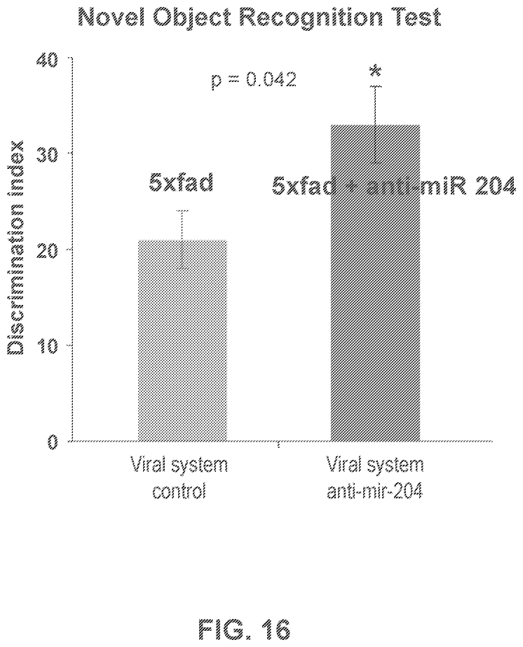

Novel object recognition Before sacrifice, the hippocampal-dependent recognition memory of treated and non-treated mice was assessed by a novel object recognition test (NORT). The first three days, each mouse was left to get used to the open field box, without any objects (10 min/session). On the fourth day, mice were left for 10 min to explore two identical objects (A+A). On the fifth day, each mouse was exposed for 10 min to a familiar object A and a novel object, namely B. After this, the objects and the open field box were cleaned with soap and water in order to avoid the presence of olfactory signs. Recorded videos were analyzed and the discrimination index (DI) was calculated dividing the exploration time of the novel object by the total exploration time [21] Exploration was defined as sniffing or touching an object. Mice with a total exploration time of <5 s for an object were removed from analyses. FIG. 16 shows that the mice received anti-miR-204-5p had a better index than the mice received a negative control. This data showed that cognition, learning, and memory of the mice received anti-miR-204 inhibitor were enhanced compared to the control mice.

### Example 5

### Effects of Anti-miR-204 Inhibitor in Secretion of Inflammatory Mediators

Cells will be in vitro treated with a negative control and an anti-miR-204 inhibitor. The cells will then be treated with an alpha-synuclein protein and will be measured to determine section of inflammatory mediators, such as TNF-α and/or IL-1β. It is expected that the inflammatory mediators will be reduced.

### Example 6

### Neuroprotective Effects of Anti-miR-204 Inhibitor by Increasing of Nurr1 Protein Expression

In order to test the neuroprotective effect of an anti-miR-204, primary mouse hippocampal cells will be treated with an anti-miR-204 and will then be reacted with amyloid beta (Aβ) or alpha-synuclein. Cell death will then be measured to determine whether the anti-miR-204 treatment can rescue cell death caused by AP or alpha-synuclein because the anti-miR-204 is expected to increase the Nurr1 protein expression. MTT assay or TUNEL assay can be used for the analysis. It is expected that the anti-miR-204 inhibitor has a neuroprotective effect.

In order to determine the cell death relationship between the Nurr1 protein and alpha-synuclein, HT22 mouse hippocampal cell line will be treated with a lentivirus comprising Nurr1 shRNAs. The Nurr1 knock down will then be measured by a cell death assay. It is expected that the cells lacking the Nurr1 protein will show increased cell deaths.

Dying microglia secrets microglial activators (MMP3, laminin, a-synuclein, neuromelanin, etc), which can induce inflammatory response. The effect of an anti-miR-204 on microglia treated with amyloid beta or alpha-synuclein will be tested. It is expected that anti-miR-204 will reduce the microglial activator secretion.

### Example 7

### Neurogenesis of Anti-miR-204 Inhibitor by Increasing Nurr1 Protein Expression

In order to determine the effect of anti-miR-204 on adult neurogenesis, neural progenitors will be separated from adult CNS and will be grown under suitable condition. The progenitor cells will be treated with anti-miR-204 and will be measured for the proliferation of the cells by BrdU ELISA. It is expected that the anti-miR-204 will increase adult neurogenesis.

### Example 8

### Duration of Efficacy of Anti-miR-204 Viral System

The duration of efficacy using anti-miR204 viral system will be determined by measuring the virus titer eight weeks, nine weeks, ten weeks, 11 weeks, or 12 weeks after administration of the anti-miR-204 viral system.

### Example 9

Relationship Between Increase of Nurr1 Protein Expression and Loss of Dendrite Spine Density

Sliced brain tissue will be stained with synaptophysin/PSD-95 antibody, a pre/postsynaptic marker, and the density of synapses will be quantitated. In this study, we will investigate the difference in synaptic densities of the hippocampus in normal control mice, saline administered mice, and mice treated with anti-miR-204 viral system. We will then examine whether the increased expression of Nurr1 protein protects against the loss of dendritic spine density.

### Example 10

### Influence of Anti-miR-204 Viral System on Cognitive Impairment

After administering anti-miR-204 expressing virus into 5×FAD mice, we will measure the reduction of cognitive function, which is the most important symptom of AD, by a Y-maze task and a contextual fear conditioning task. The cognitive abilities of the 5×FAD mice group receiving the anti-miR-204 expressing virus will be increased compared to 5×FAD mice receiving saline.

### Example 11

### Influence of Anti-miR-204 Expressing Virus on Behavioral Function Reduction

After administering anti-miR-204 expressing virus, a rotarod and pole test will be used to confirm the decline in behavioral function, the most important symptom of Parkinson's disease (PD). It is expected that behavioral function of the PD animal model group treated with anti-miR-204 expressing virus will be improved compared to saline-treated PD animal model (6-OHDA induced model, PFF induced model, MPTP induced model and hA53T alpha-synuclein TG mouse).

### Example 12

### Effect of Anti-miR-204 Expressing Virus on Neuro-Inflammatory Response

Microgliosis is known to be one of the major lesions of Parkinson's disease and Alzheimer's disease and is known to be inhibited by increased expression of a Nurr1 protein. To investigate whether microgliosis can be altered by an anti-miR-204 expressing virus, we will stain microglia using Iba1 and will quantify the results. It is expected that the hippocampus and cortex of the 5×FAD mice group treated with the anti-miR-204 expressing virus will show significantly reduced microgliosis compared to the 5×FAD mice treated with saline.

### Example 13

### Effect of Anti-miR-204 on Neuronal Damage

Nerve cell loss is known to be the most characteristic feature of Parkinson's disease and Alzheimer's disease. In order to analyze the effect of anti-miR-204, we will perform immunohistochemical staining with Neuronal nuclear antigen (NeuN) as a marker for neuron. It is expected that the anti-miR-204 administration will significantly inhibit cellular damage in the hippocampus.

### Example 14

### Analysis of Nurr1 Expression in Spinal Cord Motor Neurons of Patients with ALS

To determine whether Nurr1 expression is increased or decreased by ALS, a real-time PCR and Western blot analysis can be used. We will compare the expression of Nurr1 protein in spinal cord motor neurons in patients with ALS and normal subjects.

### Example 15

### Analysis of Nurr1 Expression in Spinal Cord Motor Neurons of Patients with Parkinson's Disease

To test the Nurr1 expression in Parkinson's disease, we will use a real-time PCR and Western blotting. It is expected that the expression of Nurr1 protein will be decreased compared to that of the normal person.

It is to be appreciated that the Detailed Description section, and not the Summary and Abstract sections, is intended to be used to interpret the claims. The Summary and Abstract sections may set forth one or more but not all exemplary embodiments of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

The claims in the instant application are different than those of the parent application or other related applications. The Applicant therefore rescinds any disclaimer of claim scope made in the parent application or any predecessor application in relation to the instant application. The Examiner is therefore advised that any such previous disclaimer and the cited references that it was made to avoid, may need to be revisited. Further, the Examiner is also reminded that any disclaimer made in the instant application should not be read into or against the parent application.

What is claimed is:

1. An miR-204 inhibitor for treating a disease or condition associated with a decreased level of a Nurr1 protein in a subject in need thereof, wherein the miR-204 inhibitor is suitable for administration to the subject.

2. An miR-204 inhibitor for increasing a Nurr1 protein expression in a cell, wherein the miR-204 inhibitor increases the Nurr1 protein expression when contacted with the cell.

3. The miR-204 inhibitor for use of claim 2, wherein the cell is present in a subject.

4. The miR-204 inhibitor for use of any one of claims 1 to 3, wherein the miR-204 inhibitor comprises a nucleotide sequence comprising at least one miR-204 binding site.

5. The miR-204 inhibitor for use of claim 4, wherein the nucleotide sequence is a vector comprising a promoter and an RNA expression region.

6. The miR-204 inhibitor for use of claim 5, wherein the RNA expression region is located downstream of the promoter, wherein the RNA expression region comprises a nucleotide sequence expressing an RNA comprising at least one miR-204 binding site, and wherein the RNA expression region does not encode a protein.

7. The miR-204 inhibitor for use of claim 5 or 6, wherein the vector does not encode a protein that is heterologous to the vector.

8. The miR-204 inhibitor for use of any one of claims 4 to 7, wherein the at least one miR-204 binding site binds to endogenous miR-204 and regulates expression of one or more endogenous polypeptides.

9. The miR-204 inhibitor for use of claim 8, wherein the at least one miR204 binding site increases expression of the Nurr1 protein.

10. The miR-204 inhibitor for use of any one of claims 1 to 9, wherein the miR204 inhibitor does not increase expression of an NMDA receptor.

11. The miR-204 inhibitor for use of claim 10, wherein the miR204 inhibitor does not increase expression of a EphB2 protein.

12. The miR-204 inhibitor for use of any one of claims 1 to 11, wherein the miR204 inhibitor increases the expression of the Nurr1 protein after the administration or contact by at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, at least about 6 fold, at least about 6.5 fold, at least about 7 fold, at least about 7.5 fold, or at least about 8 fold compared to the expression prior to the administration or contact.

13. The miR-204 inhibitor for use of any one of claims 1 to 12, wherein the miR204 inhibitor treats a disease or condition associated with a decreased expression of the Nurr1 protein, but not with a decreased expression of an NMDA receptor and/or an EphB2 protein.

14. The miR-204 inhibitor for use of any one of claims 1 to 13, wherein the disease or condition is not associated with a decreased hippocampus function.

15. The miR-204 inhibitor for use of claim 13 or 14, wherein the disease or condition is Alzheimer disease.

16. The miR-204 inhibitor for use of any one of claims 1, 13, and 14, wherein the disease or condition is Parkinson's disease, prion disease, motor neuron disease, Huntington's disease, spinocerebellar ataxia, spinal muscular atrophy, amyotrophic lateral sclerosis, or any combination thereof.

17. The miR-204 inhibitor for use of any one of claims 1 to 16, wherein the at least one miR-204 binding site hybridizes to miR-204-5p.

18. The miR-204 inhibitor for use of any one of claims 4 to 17, wherein the at least one miR-204 binding site is fully complementary to miR-204-5p.

19. The miR-204 inhibitor for use of claim 17 or 18, wherein the miR-204-5p comprises the nucleotide sequence as set forth in SEQ ID NO: 1.

20. The miR-204 inhibitor for use of any one of claims 4 to 19, wherein the at least one miR-204 binding site comprises the nucleic acid sequence set forth in SEQ ID NO: 2.

21. The miR-204 inhibitor for use of any one of claims 5 to 20, wherein the nucleotide sequence expressing the RNA comprises the nucleic acid sequence set forth in SEQ ID NO: 3.

22. The miR-204 inhibitor for use of any one of claims 4 to 16, wherein the at least one miR-204 binding site hybridizes to miR-204-3p.

23. The miR-204 inhibitor for use of claim 22, wherein the at least one miR-204 binding site is fully complementary to miR-204-3p.

24. The miR-204 inhibitor for use of claim 22 or 23, wherein the miR-204-3p comprises the nucleotide sequence as set forth in SEQ ID NO: 5.

25. The miR-204 inhibitor for use of any one of claims 22 to 24, wherein the at least one miR-204 binding site comprises the nucleic acid sequence set forth in SEQ ID NO: 6.

26. The miR-204 inhibitor for use of any one of claims 22 to 25, wherein the nucleotide sequence expressing the RNA comprises the nucleic acid sequence set forth in SEQ ID NO: 7.

27. The miR-204 inhibitor for use of any one of claims 5 to 26, wherein the RNA comprises at least two miR-204 binding sites.

28. The miR-204 inhibitor for use of claim 27, wherein the RNA comprises two miR-204 binding sites, three miR-204 binding sites, four miR-204 binding sites, five miR-204 binding sites, or six miR-204 binding sites.

29. The miR-204 inhibitor for use of claim 27, wherein the RNA comprises two miR-204 binding sites.

30. The miR-204 inhibitor for use of any one of claims 4 to 29, wherein each of the at least one miR-204 binding site comprises the nucleic acid sequence set forth in SEQ ID NO:19 at the 5′ end.

31. The miR-204 inhibitor for use of any one of claims 4 to 30, wherein each of the at least one miR-204 binding site comprises the nucleic acid sequence set forth in SEQ ID NO:20 at the 3′ end.

32. The miR-204 inhibitor for use of claim 29, wherein the two miR-204 binding sites comprise a nucleotide sequence forming a loop in between the miR-204 binding sites.

33. The miR-204 inhibitor for use of claim 32, wherein the loop comprises a nucleotide sequence comprising the nucleic acid sequence set forth in SEQ ID NO:13.

34. The miR-204 inhibitor for use of any one of claims 29 to 33, wherein the RNA comprising the two miR-204 binding sites comprises a first stem region and a second stem region.

35. The miR-204 inhibitor for use of claim 34, wherein the first stem region comprises a nucleotide sequence set forth in SEQ ID NO:9 or its complementary nucleotide sequence set forth in SEQ ID NO:11, which is linked to at least one of the two miR-204 binding sites.

36. The miR-204 inhibitor for use of 34 or 35, wherein the second stem region comprises a nucleotide sequence of set forth in SEQ ID NO:15 or its complementary nucleotide sequence set forth in SEQ ID NO:17, which is linked to at least one of the two miR-204 binding sites.

37. The miR-204 inhibitor for use of any one of claims 5 to 36, wherein the RNA comprises the nucleic acid sequence set forth in SEQ ID NO: 23, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57.

38. The miR-204 inhibitor for use of any one of claims 1 to 37, which is a virus, a plasmid, or a phagemid.

39. The miR-204 inhibitor for use of any one of claims 1 to 38, which is a virus.

40. The miR-204 inhibitor for use of claim 39, wherein the virus is selected from the group consisting of a retrovirus, a lentivirus, an adenovirus, an adeno-associated virus (AAV), an SV40-type viruse, a polyomavirus, an Epstein-Barr virus, a papilloma viruses, a herpes virus, a vaccinia virus, a polio virus, and an RNA virus.

41. The miR-204 inhibitor for use of any one of claims 1 to 40, which is an AAV.

42. The miR-204 inhibitor for use of claim 41, wherein the AAV is selected from the group consisting of AAV type 1, AAV type 2, AAV type 3A, AAV type 3B, AAV type 4, AAV type 5, AAV type 6, AAV type 7, AAV type 8, AAV type 9, AAV type 10, AAV type 11, AAV type 12, AAV type 13, snake AAV, avian AAV, bovine AAV, canine AAV, equine AAV, ovine AAV, goat AAV, shrimp AAV, and a derivative thereof.

43. The miR-204 inhibitor for use of any one of claims 5 to 42, wherein the promoter is an RNA Pol III promoter.

44. The miR-204 inhibitor for use of claim 43, wherein the RNA Pol III promoter is selected from the group consisting of the U6 promoter, the H1 promoter, the 7SK promoter, the 5S promoter, the adenovirus 2 (Ad2) VAI promoter, and any combination thereof.

45. The miR-204 inhibitor for use of any one of claims 5 to 44, wherein the promoter comprises the U6 promoter.

46. The miR-204 inhibitor for use of any one of claims 5 to 43, wherein the promoter is a constitutive promoter.

47. The miR-204 inhibitor for use of claim 46, wherein the constitutive promoter is selected from the group consisting of hypoxanthine phosphoribosyl transferase (HPRT), adenosine deaminase, pyruvate kinase, beta-actin promoter, cytomegalovirus (CMV), simian virus (e.g., SV40), papilloma virus, adenovirus, human immunodeficiency virus (HIV), Rous sarcoma virus, a retrovirus long terminal repeat (LTR), and the thymidine kinase promoter of herpes simplex virus.

48. The miR-204 inhibitor for use of any one of claims 5 to 43, wherein the promoter is an inducible promoter.

49. The miR-204 inhibitor for use of claim 48, wherein the inducible promoter is a tissue specific promoter.

50. The miR-204 inhibitor for use of claim 49, wherein the tissue specific promoter drives transcription of the coding region in a neuron, a glial cell, or in both a neuron and a glial cell.

51. The miR-204 inhibitor for use of any one of claims 1 to 50, wherein the miR204 inhibitor is formulated with a pharmaceutically acceptable carrier in a pharmaceutical composition.

52. The miR-204 inhibitor for use of any one of claims 1 and 4 to 51, wherein the administering improves one or more cognitive symptom in the subject, relative to the cognitive symptom in the subject prior to the administering.

53. The miR-204 inhibitor for use of any one of claims 1 and 4 to 52, wherein the administering reduces memory loss in the subject, relative to the memory loss in the subject prior to the administering.

54. The miR-204 inhibitor for use of any one of claims 1 and 4 to 53, wherein the administering improves memory retention in the subject, relative to the memory retention in the subject prior to the administering.

55. The miR-204 inhibitor for use of any one of claims 1 and 4 to 54, wherein the administering reduces an amyloid beta (Aβ) plaque load in the subject, relative to the amyloid beta (Aβ) plaque load in the subject prior to the administering.

56. The miR-204 inhibitor for use of any one of claims 1 and 4 to 55, wherein the administering increases dendritic spine density of a neuron in the subject, relative to the dendritic spine density of a neuron in the subject prior to the administering.

57. The miR-204 inhibitor for use of any one of claims 1 and 4 to 56, wherein the administering is via intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

58. A polynucleotide sequence comprising a nucleotide sequence at least about 80%, at least about 85%, at least about 90%, at least about 95%, at least about 96%, at least about 97%, at least about 98%, at least about 99%, or about 100% identical to the nucleotide sequence as set forth in SEQ ID NO: 27, 28, 29, 30, 31, 32, 33, 34, 35, 36, 37, 38, 39, 40, 41, 42, 43, 44, 45, 46, 47, 48, 49, 50, 51, 52, 53, 54, 55, or 56, wherein the nucleotide sequence is capable of inhibiting miR-204-5p.

59. The polynucleotide sequence of claim 58, wherein the miR-204-5p comprises SEQ ID NO: 1.

60. The polynucleotide sequence of claim 58 or 59, wherein the nucleotide sequence hybridizes SEQ ID NO: 1.

61. A polynucleotide sequence of any one of claims 58 to 60 for increasing a Nurr1 protein in a subject in need thereof, wherein the polynucleotide sequence is suitable for administration to the subject.

62. The polynucleotide sequence of claim 61, wherein the administering treats a disease or condition associated with the increased level of the Nurr1 protein.
